SEQUENCE LISTING (7 sequences)

SEQ ID NO: 1 - protein, 845 aa, Human gammaherpesvirus 8
MTPRSRLATLGTVILLVCFCAGAAHSRGDTFQTSSSPTPPGSSSKAPTKPGEEASGPKSVDFYQFRVCSASITGELFRFNLEQTCPDTKDKYHQEGILLVYKKNIVPHIFKVRRYRKIATSVTVYRGLTESAITNKYELPRPVPLYEISHMDSTYQCFSSMKVNVNGVENTFTDRDDVNTTVFLQPVEGLTDNIQRYFSQPVIYAEPGWFPGIYRVRTTVNCEIVDMIARSAEPYNYFVTSLGDTVEVSPFCYNESSCSTTPSNKNGLSVQVVLNHTVVTYSDRGTSPTPQNRIFVETGAYTLSWASESKTTAVCPLALWKTFPRSIQTTHEDSFHFVANEITATFTAPLTPVANFTDTYSCLTSDINTTLNASKAKLASTHVPNGTVQYFHTTGGLYLVWQPMSAINLTHAQGDSGNPTSSPPPSASPMTTSASRRKRRSASTAAAGGGGSTDNLSYTQLQFAYDKLRDGINQVLEELSRAWCREQVRDNLMWYELSKINPTSVMTAIYGRPVSAKFVGDAISVTECINVDQSSVNIHKSLRTNSKDVCYARPLVTFKFLNSSNLFTGQLGARNEIILTNNQVETCKDTCEHYFITRNETLVYKDYAYLRTINTTDISTLNTFIALNLSFIQNIDFKAIELYSSAEKRLASSVFDLETMFREYNYYTHRLAGLREDLDNTIDMNKERFVRDLSEIVADLGGIGKTVVNVASSVVTLCGSLVTGFINFIKHPLGGMLMIIIVIAIILIIFMLSRRTNTIAQAPVKMIYPDVDRRAPPSGGAPTREEIKNILLGMHQLQQEERQKADDLKKSTPSVFQRTANGLRQRLRGYKPLTQSLDISPETGE

SEQ ID NO: 2 - protein, 1376 aa, Human gammaherpesvirus 8
MEATLEQRPFPYLATEANLLTQIKESAADGLFKSFQLLLGKDAREGSVRFEALLGVYTNVVEFVKFLETALAAACVNTEFKDLRRMIDGKIQFKISMPTIAHGDGRRPNKQRQYIVMKACNKHHIGAEIELAAADIELLFAEKETPLDFTEYAGAIKTITSALQFGMDALERGLVDTVLAVKLRHAPPVFILKTLGDPVYSERGLKKAVKSDMVSMFKAHLIEHSFFLDKAELMTRGKQYVLTMLSDMLAAVCEDTVFKGVSTYTTASGQQVAGVLETTDSVMRRLMNLLGQVESAMSGPAAYASYVVRGANLVTAVSYGRAMRNFEQFMARIVDHPNALPSVEGDKAALADGHDEIQRTRIAASLVKIGDKFVAIESLQRMYNETQFPCPLNRRIQYTYFFPVGLHLPVPRYSTSVSVRGVESPAIQSTETWVVNKNNVPLCFGYQNALKSICHPRMHNPTQSAQALNQAFPDPDGGHGYGLRYEQTPNMNLFRTFHQYYMGKNVAFVPDVAQKALVTTEDLLHPTSHRLLRLEVHPFFDFFVHPCPGARGSYRATHRTMVGNIPQPLAPREFQESRGAQFDAVTNMTHVIDQLTIDVIQETAFDPAYPLFCYVIEAMIHGQEEKFVMNMPLIALVIQTYWVNSGKLAFVNSYHMVRFICTHMGNGSIPKEAHGHYRKILGELIALEQALLKLAGHETVGRTPITHLVSALLDPHLLPPFAYHDVFTDLMQKSSRQPIIKIGDQNYDNPQNRATFINLRGRMEDLVNNLVNIYQTRVNEDHDERHVLDVAPLDENDYNPVLEKLFYYVLMPVCSNGHMCGMGVDYQNVALTLTYNGPVFADVVNAQDDILLHLENGTLKDILQAGDIRPTVDMIRVLCTSFLTCPFVTQAARVITKRDPAQSFATHEYGKDVAQTVLVNGFGAFAVADRSREAAETMFYPVPFNKLYADPLVAATLHPLLANYVTRLPNQRNAVVFNVPSNLMAEYEEWHKSPVAAYAASCQATPGAISAMVSMHQKLSAPSFICQAKHRMHPGFAMTVVRTDEVLAEHILYCSRASTSMFVGLPSVVRREVRSDAVTFEITHEIASLHTALGYSSVIAPAHVAAITTDMGVHCQDLFMIFPGDAYQDRQLHDYIKMKAGVQTGSPGNRMDHVGYTAGVPRCENLPGLSHGQLATCEIIPTPVTSDVAYFQTPSNPRGRAACVVSCDAYSNESAERLLYDHSIPDPAYECRSTNNPWASQRGSLGDVLYNITFRQTALPGMYSPCRQFFHKEDIMRYNRGLYTLVNEYSARLAGAPATSTTDLQYVVVNGTDVFLDQPCHMLQEAYPTLAASHRVMLDEYMSNKQTHAPVHMGQYLIEEVAPMKRLLKLGNKVVY

SEQ ID NO: 3 - protein, 550 aa, Human gammaherpesvirus 8
MAFLRQTLWILWTFTMVIGQDNEKCSQKTLIGYRLKMSRDGDIAVGETVELRCRSGYTTYARNITATCLQGGTWSEPTATCNKKSCPNPGEIQNGKVIFHGGQDALKYGANISYVCNEGYFLVGREYVRYCMIGASGQMAWSSSPPFCEKEKCHRPKIENGDFKPDKDYYEYNDAVHFECNEGYTLVGPHSIACAVNNTWTSNMPTCELAGCKFPSVTHGYPIQGFSLTYKHKQSVTFACNDGFVLRGSPTITCNVTEWDPPLPKCVLEDIDDPNNSNPGRLHPTPNEKPNGNVFQRSNYTEPPTKPEDTHTAATCDTNCEQPPKILPTSEGFNETTTSNTITKQLEDEKTTSQPNTHITSALTSMKAKGNFTNKTNNSTDLHIASTPTSQDDATPSIPSVQTPNYNTNAPTRTLTSLHIEEGPSNSTTSEKATASTLSHNSHKNDTGGIYTTLNKTTQLPSTNKPTNSQAKSSTKPRVETHNKTTSNPAISLTDSADVPQRPREPTLPPIFRPPASKNRYLEKQLVIGLLTAVALTCGLITLFHYLFFR

SEQ ID NO: 4 - DNA, 20 bp, synthetic construct
  source 1..20: mol_type = other DNA
cgcaatgcgttacgttgttg

SEQ ID NO: 5 - DNA, 18 bp, synthetic construct
  source 1..18: mol_type = other DNA
gcccggactgttgaatcg

SEQ ID NO: 6 - DNA, 21 bp, synthetic construct
  source 1..21: mol_type = other DNA
catgtacgttgctatccaggc

SEQ ID NO: 7 - DNA, 21 bp, synthetic construct
  source 1..21: mol_type = other DNA
ctccttaatgtcacgcacgat

(19) United States
(12) Patent Application Publication
(10) Pub. No.: US 2024/0123059 A1
(43) Pub. Date: Apr. 18, 2024

(54) Kaposi's Sarcoma Associated Herpesvirus Vaccine and Methods of Making and Using Thereof

(71) Applicant: The Regents of the University of California, Oakland, CA (US)

(72) Inventors: Ting-Ting Wu, Los Angeles, CA (US); Alex Lam, Rosemead, CA (US); Caius Radu, Los Angeles, CA (US)

(21) Appl. No.: 18/487,526

(22) Filed: Oct. 16, 2023

Related U.S. Application Data
(60) Provisional application No. 63/416,685, filed on Oct. 17, 2022.

(51) Int. Cl. A61K 39/245; A61K 39/39; C07K 16/08

(52) U.S. Cl. CPC A61K 39/245; A61K 39/39; C07K 16/085; A61K 2039/5258

(57) ABSTRACT

Disclosed herein are compositions and methods for inducing and/or enhancing complement-mediated neutralization of a herpesvirus.

### CROSS-REFERENCE TO RELATED APPLICATIONS

This application claims the benefit of U.S. Patent Application No. 63/416,685, filed Oct. 17, 2022, which is herein incorporated by reference in its entirety.

### ACKNOWLEDGEMENT OF GOVERNMENT SUPPORT

This invention was made with Government support under Grant Number DE028774, awarded by the National Institutes of Health. The Government has certain rights in the invention.

### REFERENCE TO A SEQUENCE LISTING SUBMITTED VIA EFS-WEB

The content of the XML text file of the sequence listing named “20231016_034044_246US1_ST26” which is 10,002 bases in size was created on Oct. 16, 2023 and electronically submitted via PatentCenter herewith the application is incorporated herein by reference in its entirety.

### BACKGROUND OF THE INVENTION

### 1. Field of the Invention

The field generally relates to Kaposi's Sarcoma Associated Herpesvirus and vaccines and antibodies thereto.

### 2. Description of the Related Art

Kaposi's Sarcoma Associated Herpesvirus (KSHV or Human gammaherpesvirus 8) is the etiological agent for cancers such as Kaposi's Sarcoma (KS) and primary effusion lymphoma. KS, the most frequent malignancy associated with KSHV infection, is estimated to account for about 34,000 new cases and 15,000 deaths annually worldwide. KS malignancies manifest as lesions composed of endothelial cells on the skin, lymph nodes, lungs, and digestive tract. While the occurrence of KS is low overall in the US at a rate of 4.5 cases per million people in 2017, the occurrence may be up to 500 times higher in transplant patients and in people living with Human Immunodeficiency Virus (HIV).

Vaccination against KSHV would reduce the disease burden associated with infection. Thus, a need exists for a vaccine against KSHV.

### SUMMARY OF THE INVENTION

In some embodiments, the present invention is directed to a mutant herpesvirus that is having a mutation in its glycoprotein B (gB). In some embodiments, the mutant is capsid deficient. In some embodiments, the mutant is deficient in the major capsid protein. In some embodiments, the gB of the mutant herpesvirus has a sequence that, when optimally aligned with Accession No. YP_001129354.1 (SEQ ID NO: 1), has an amino acid other than aspartic acid (D) at position 470 and/or an amino acid other than glycine (G) at position 471. In some embodiments, the gB of the mutant herpesvirus has a sequence that, when optimally aligned with Accession No. YP_001129354.1, has a proline (P) at position 470 and/or position 471. In some embodiments, the gB protein of the mutant herpesvirus has (1) a sequence that is at least 95%, at least 96%, at least 97%, at least 98%, or at least 99% sequence identity to Accession No. YP_001129354.1 and (2) an amino acid other than aspartic acid (D) at position 470 and/or an amino acid other than glycine (G) at position 471 when optimally aligned thereto. In some embodiments, the gB protein of the mutant herpesvirus has (1) a sequence that is at least 95%, at least 96%, at least 97%, at least 98%, or at least 99% sequence identity to Accession No. YP_001129354.1 and (2) a proline (P) at position 470 and/or position 471 when optimally aligned thereto. In some embodiments, the herpesvirus is a Kaposi's Sarcoma Associated Herpesvirus (KSHV). In some embodiments, the capsid deficient mutant lacks an ORF25 gene or its ORF25 gene encodes an ORF25 protein that lacks at least 25 amino acid residues at the N-terminus as compared to the ORF25 wildtype sequence. In some embodiments, the ORF25 protein has a mutation selected from ORF25Δ30, ORF25Δ31, ORF25Δ32, ORF25Δ33, ORF25Δ34, ORF25Δ35, ORF25Δ36, ORF25Δ37, ORF25Δ38, ORF25Δ39, ORF25Δ40, ORF25Δ41, ORF25Δ42, ORF25Δ43, ORF25Δ44, ORF25Δ45, ORF25Δ46, ORF25Δ47, ORF25Δ48, ORF25Δ49, ORF25Δ50, ORF25Δ51, ORF25Δ52, ORF25Δ53, ORF25Δ54, ORF25Δ55, ORF25Δ56, ORF25Δ57, ORF25Δ58, ORF25Δ59, ORF25Δ60, ORF25Δ61, ORF25Δ62, ORF25Δ63, ORF25Δ64, and ORF25Δ65 as compared to the ORF25 wildtype sequence. In some embodiments, the protein comprises or consists of amino acid residues 24-314 of the ORF4 of the herpesvirus. In some embodiments, the ORF4 of the herpesvirus contains at least 95%, at least 96%, at least 97%, at least 98%, at least 99%, or 100% sequence identity to the ORF4 wildtype sequence of KSHV. In some embodiments, the present invention is directed to a composition comprising, consisting essentially of, or consisting of the mutant herpesvirus and an adjuvant. In some embodiments, the adjuvant is a CpG oligonucleotide (CpG ODN) that is an agonist of toll like receptor 9 (TLR9), an empty lipid nanoparticle (LNP), or a polyU oligonucleotide encapsulated in a lipid nanoparticle (polyUs LNP). In some embodiments, the polyU oligonucleotide (a) is 10-25 bases long; and/or (b) has phosphorothioate linkages. In some embodiments, the adjuvant is a 21-mer phosphorothioate-linked polyU oligonucleotide.

In some embodiments, the present invention is directed to a composition for inducing and/or enhancing complement-mediated neutralization of a herpesvirus which comprises, consists essentially of, or consists of (1)(a) a mutant herpesvirus which is a capsid deficient mutant having a deletion in its major capsid protein as compared to the wildtype major capsid protein, and/or a gB mutant that has mutation in its glycoprotein B (gB); (b) virus-like vesicles made from the mutant herpesvirus, which is capsid deficient; (c) a protein that comprises, consists essentially of, or consists of amino acid residues 24-150 of an ORF4 protein of the herpesvirus or a nucleic acid molecule that encodes the protein; and/or (d) an antibody raised against (a), (b), or (c), or a synthetic antibody derived from the antibody; and (2) an adjuvant. In some embodiments, the mutant herpesvirus is a mutant of the given parental herpesvirus. In some embodiments, the composition comprising the virus-like vesicles is free of infectious virions. In some embodiments, the mutant herpesvirus is both a capsid deficient mutant and a gB mutant. In some embodiments, the gB of the mutant herpesvirus has a sequence that, when optimally aligned with Accession No. YP_001129354.1, has an amino acid other than aspartic acid (D) at position 470 and/or an amino acid other than glycine (G) at position 471. In some embodiments, the gB of the mutant herpesvirus has a sequence that, when optimally aligned with Accession No. YP_001129354.1, has a proline (P) at position 470 and/or position 471. In some embodiments, the gB protein of the mutant herpesvirus has (1) a sequence that is at least 95%, at least 96%, at least 97%, at least 98%, or at least 99% sequence identity to Accession No. YP_001129354.1 and (2) an amino acid other than aspartic acid (D) at position 470 and/or an amino acid other than glycine (G) at position 471 when optimally aligned thereto. In some embodiments, the gB protein of the mutant herpesvirus has (1) a sequence that is at least 95%, at least 96%, at least 97%, at least 98%, or at least 99% sequence identity to Accession No. YP_001129354.1 and (2) a proline (P) at position 470 and/or position 471 when optimally aligned thereto. In some embodiments, the adjuvant is a CpG oligonucleotide (CpG ODN) that is an agonist of toll like receptor 9 (TLR9), an empty lipid nanoparticle (LNP), or a polyU oligonucleotide encapsulated in a lipid nanoparticle (polyUs LNP). In some embodiments, the polyU oligonucleotide (a) is 10-25 bases long; and/or (b) has phosphorothioate linkages. In some embodiments, the adjuvant is a 21-mer phosphorothioate-linked polyU oligonucleotide. In some embodiments, the herpesvirus is a Kaposi's Sarcoma Associated Herpesvirus (KSHV). In some embodiments, the capsid deficient mutant lacks an ORF25 gene or its ORF25 gene encodes an ORF25 protein that lacks at least 25 amino acid residues at the N-terminus as compared to the ORF25 wildtype sequence. In some embodiments, the ORF25 protein has a mutation selected from ORF25Δ30, ORF25Δ31, ORF25Δ32, ORF25Δ33, ORF25Δ34, ORF25Δ35, ORF25Δ36, ORF25Δ37, ORF25Δ38, ORF25Δ39, ORF25Δ40, ORF25Δ41, ORF25Δ42, ORF25Δ43, ORF25Δ44, ORF25Δ45, ORF25Δ46, ORF25Δ47, ORF25Δ48, ORF25Δ49, ORF25Δ50, ORF25Δ51, ORF25Δ52, ORF25Δ53, ORF25Δ54, ORF25Δ55, ORF25Δ56, ORF25Δ57, ORF25Δ58, ORF25Δ59, ORF25Δ60, ORF25Δ61, ORF25Δ62, ORF25Δ63, ORF25Δ64, and ORF25Δ65 as compared to the ORF25 wildtype sequence. In some embodiments, the protein comprises or consists of amino acid residues 24-314 of the ORF4 of the herpesvirus. In some embodiments, the ORF4 of the herpesvirus contains at least 95%, at least 96%, at least 97%, at least 98%, at least 99%, or 100% sequence identity to the ORF4 wildtype sequence of KSHV. In some embodiments, the herpesvirus comprises a gB protein having at least 95%, at least 96%, at least 97%, at least 98%, at least 99%, or 100% sequence identity to Accession No. YP_001129354.1 (SEQ ID NO: 1), a major capsid protein having at least 95%, at least 96%, at least 97%, at least 98%, at least 99%, or 100% sequence identity to Accession No. YP_001129378.1 (SEQ ID NO: 2), and/or an ORF4 protein having at least 95%, at least 96%, at least 97%. at least 98%, at least 99%, or 100% sequence identity to Accession No. YP_001129351.1 (SEQ ID NO: 3), and the mutant herpesvirus is a mutant of the herpesvirus.

In some embodiments, the present invention is directed to a method of making an antibody against a herpesvirus, which comprises immunizing a subject with (1) a mutant herpesvirus, virus-like vesicles, and/or ORF4 proteins as described herein, alone, or (2) in combination with an adjuvant. In some embodiments, the antibody is a complement-mediated neutralizing antibody.

In some embodiments, the present invention is directed to sera comprising antibodies against a herpesvirus made by methods described herein.

In some embodiments, the present invention is directed to a method of immunizing a subject against infection by a herpesvirus, which comprises administering to the subject (1) a mutant herpesvirus, virus-like vesicles, and/or ORF4 proteins as described herein, alone, or (2) in combination with an adjuvant.

In some embodiments, the present invention is directed to a method of inducing or enhancing an immune response against a herpesvirus in a subject, which comprises administering to the subject (1) a mutant herpesvirus, virus-like vesicles, and/or ORF4 proteins as described herein, alone, or (2) in combination with an adjuvant. In some embodiments, the immune response is complement-mediated neutralization of the herpesvirus.

In some embodiments, the present invention is directed to a method of inhibiting or treating a herpesvirus infection and/or a disease caused by the herpesvirus infection in a subject, which comprises administering to the subject (1) a mutant herpesvirus, virus-like vesicles, and/or ORF4 proteins as described herein, alone, or in combination with an adjuvant, or (2) antibodies raised thereagainst. In some embodiments, the disease is a cancer such as Kaposi's Sarcoma (KS) or primary effusion lymphoma.

Both the foregoing general description and the following detailed description are exemplary and explanatory only and are intended to provide further explanation of the invention as claimed. The accompanying drawings are included to provide a further understanding of the invention and are incorporated in and constitute part of this specification, illustrate several embodiments of the invention, and together with the description explain the principles of the invention.

### DESCRIPTION OF THE DRAWINGS

This invention is further understood by reference to the drawings wherein:

FIG. 1-FIG. 3: ΔVLVs present KSHV antigens without viral DNA or infectivity. Cryo-electron micrographs of ΔVLVs and KSHV virions were obtained from reactivated iSLK-25Δ60 cells or iSLK-WT cells (not shown). Negative stain micrographs of ΔVLVs and KSHV virions labeled with K8.1 antibodies and immunogold beads were obtained (not shown). Vesicles were considered positively labeled if they were over 100 nm in diameter and were labeled with more than five gold beads.

FIG. 1: A volcano plot showing proteins found in ΔVLVs and KSHV virion preparations. Differentially present (log 2 fold-change<−2 and p<0.05) proteins are marked with triangles and other viral proteins of interest are marked with squares.

FIG. 2: Infectious virus titer from ΔVLVs and KSHV preparations or preparations inactivated with betapropiolactone (BPL).

FIG. 3: Ratio of viral ORF50 DNA in ΔVLVs and KSHV preparations to actin from carrier DNA used during isolation. Statistical analysis: Ordinary one-way ANOVA with Dunnett's (c) or Tukey's (d) test for multiple comparisons. *p<0.05, **p<0.01, ****p<0.0001. Mean and standard deviation shown. N=3 independent infection assays (d) or DNA extractions (e).

FIG. 4-FIG. 6: Mice immunized intramuscularly with adjuvanted ΔVLVs generate virus-specific antibody responses.

FIG. 4: Immunization scheme to study immunogenicity of ΔVLVs with adjuvant used in FIG. 5. Immunofluorescence images of pooled VLV+polyUs-LNP immune serum at a 1:250 dilution binding to uninduced or reactivated iSLK-WT cells were obtained (not shown).

FIG. 5: Endpoint K8.1 ELISA titers of ΔVLV sera. LOD=limit of detection. Statistical analysis: Ordinary one-way ANOVA with Dunnett's test for multiple comparisons. **p<0.01. Mean with standard deviation shown. N=3-4 per group.

FIG. 6: Immunization scheme to compare VLV+BPL and KSHV+BPL with polyUs-LNP adjuvant used in for immunofluorescence and heatmap studies. Immunofluorescence images of pooled mock or VLV+BPL immune serum at the indicated dilutions binding to 293T cells expressing KSHV glycoproteins were obtained (not shown).

FIG. 7 and FIG. 8: Immunization with adjuvanted ΔVLVs generates virus-specific T cells.

FIG. 7: Mice were immunized as shown in FIG. 4. IFNγ secreting cell frequencies from ΔVLVs-immunized mouse splenocytes restimulated with ΔVLVs or KSHV+BPL. Ordinary one-way ANOVA with Dunnett's test for multiple comparisons. FIG. 5: Mice were immunized as shown in FIG. 6. Percentages of AIM-positive CD4 and CD8 cells after subtracting background from unstimulated cells. Two-way ANOVA with Tukey's test for multiple comparisons. *p<0.05, ** p<0.01, ***p<0.001, ****p<0.0001. Mean and standard deviation are shown. N=4 mice per group.

FIG. 9 and FIG. 10: ΔVLV sera from mice neutralize KSHV infection. 50% neutralization titers of ΔVLV sera from mice immunized as shown in FIG. 4. Infection was performed using soluble virus (FIG. 9) or spin infection (FIG. 10). LOD=limit of detection. Statistical analysis: Ordinary one-way ANOVA with Tukey's test for multiple comparisons. *p<0.05. Median is shown. N=3-4 mouse serum samples per group.

FIG. 11-FIG. 14: Complement-mediated enhancement of neutralization by ΔVLV sera depends on antibodies targeting ORF4. Mice were immunized according to the scheme in FIG. 6 and serum from the VLV+BPL group were serially diluted for neutralization.

FIG. 11: Spin infection neutralization by ΔVLV sera in the presence or absence of complement.

FIG. 12: Complement-mediated spin infection neutralization by ΔVLV sera adsorbed on 293T cells or 293T cells expressing K8.1 or ORF4.

FIG. 13: Complement-mediated spin infection neutralization by ΔVLV sera on WT KSHV or KSHV ORF4-dSCR.

FIG. 14: Complement-mediated spin infection neutralization by ΔVLV sera adsorbed on 293T cells or 293T cells expressing ORF4, ORF4-dSCR, or ORF4-SCRonly. Statistical analysis: Two-way ANOVA with Tukey's multiple comparisons test. Differences between ΔVLVs with and without complement (FIG. 11), between 293T-adsorbed and 293T-ORF4 adsorbed (FIG. 12), between ΔVLV sera neutralization of WT KSHV and ORF4-dSCR mutant (FIG. 13), or between 293T-adsorbed and 293T-ORF4-SCRonly adsorbed (FIG. 14) are shown. Mean and standard deviation are shown. N=3-4 mice per group.

FIG. 15 and FIG. 16: Serum from Kaposi-Sarcoma positive (KS+) human patients does not possess complement-enhanced neutralization. Neutralization of WT KSHV by serum diluted 1:100 in the presence and absence of normal human serum (NHS) as a source of complement (left) and difference in neutralization upon addition of complement (right). Samples are separated by K8.1 (FIG. 15) or ORF4 (FIG. 16) serostatus as determined by ELISA. Statistical analysis: 2-way ANOVA with Sidak's multiple comparison's test (% neutralization) or Welch's t test (change in neutralization). **p<0.05. Mean and standard deviation shown. N=13 (K8.1−), 26 (K8.1+), 35 (ORF4−), or 3 (ORF4+) human serum samples.

FIG. 17 and FIG. 18: Antibody responses after intraperitoneal immunization of ΔVLVs.

FIG. 17: Immunization scheme for FIG. 18 and heatmaps (not shown).

FIG. 18: K8.1 total IgG binding ELISA. Two-way ANOVA with Tukey's multiple comparisons. Difference between VLV+BPL and KSHV+BPL is shown. **p<0.01. Mean and standard deviation are shown. N=3-4 mice per group

FIG. 19: Antibody responses after intramuscular immunization of adjuvanted ΔVLVs. Mice were immunized as shown in FIG. 6. K8.1 ELISA signals from mice immunized with VLV+BPL and KSHV+BPL. Mean and standard deviation are shown. N=3-4 mice per group. Immunofluorescence images of 293T cells expressing KSHV glycoproteins or SARS-CoV-2 Spike stained with pooled mock or VLV+BPL+polyUs-LNPs immune serum at a 1:100 dilution were also obtained (not shown).

FIG. 20: Antibodies raised against mutant gB virions exhibit substantially similar complement-dependent neutralization of herpesviruses as antibodies raised against chemically inactivated wildtype virions. gB virion=KSHV mutants having mutation in their gB protein; WT virion=chemically inactivated wildtype KSHV; NHS=Normal Human Serum (complement source); +=presence of NHS; −=absence of NHS.

FIG. 21 is Table 1.

FIG. 22 is Table 2.

### DETAILED DESCRIPTION OF THE INVENTION

Disclosed herein are compositions for inhibiting and/or treating infections and diseases caused by herpesviruses such as Kaposi's Sarcoma-Associated Herpesvirus (KSHV) and methods of making and using thereof. Also disclosed are methods and compositions for enhancing complement-mediated neutralization of herpesviruses such as KSHV.

The genomes of all herpesviruses contain genes that encode a major capsid protein, a small capsid protein, and glycoprotein B. As such, the methods and compositions described herein are applicable to all herpesviruses. Thus, in some embodiments, the herpesvirus comprises a gB protein having at least 95%, at least 96%, at least 97%, at least 98%, at least 99%, or 100% sequence identity to Accession No. YP_001129354.1 (SEQ ID NO: 1). In some embodiments, the herpesvirus comprises a major capsid protein having at least 95%, at least 96%, at least 97%, at least 98%, at least 99%, or 100% sequence identity to Accession No. YP_001129378.1 (SEQ ID NO: 2). In some embodiments, the herpesvirus comprises an ORF4 protein having at least 95%, at least 96%, at least 97%, at least 98%, at least 99%, or 100% sequence identity to Accession No. YP_001129351.1 (SEQ ID NO: 3). In some embodiments, the herpesvirus comprises a gB protein having at least 95%, at least 96%, at least 97%, at least 98%, at least 99%, or 100% sequence identity to Accession No. YP_001129354.1 (SEQ ID NO: 1) and a major capsid protein having at least 95%, at least 96%, at least 97%, at least 98%, at least 99%, or 100% sequence identity to Accession No. YP_001129378.1 (SEQ ID NO: 2). In some embodiments, the herpesvirus comprises a gB protein having at least 95%, at least 96%, at least 97%, at least 98%, at least 99%, or 100% sequence identity to Accession No. YP_001129354.1 (SEQ ID NO: 1) and an ORF4 protein having at least 95%, at least 96%, at least 97%, at least 98%, at least 99%, or 100% sequence identity to Accession No. YP_001129351.1 (SEQ ID NO: 3). In some embodiments, the herpesvirus comprises a major capsid protein having at least 95%, at least 96%, at least 97%, at least 98%, at least 99%, or 100% sequence identity to Accession No. YP_001129378.1 (SEQ ID NO: 2) and an ORF4 protein having at least 95%, at least 96%, at least 97%, at least 98%, at least 99%, or 100% sequence identity to Accession No. YP_001129351.1 (SEQ ID NO: 3). In some embodiments, the herpesvirus comprises a gB protein having at least 95%, at least 96%, at least 97%, at least 98%, at least 99%, or 100% sequence identity to Accession No. YP_001129354.1 (SEQ ID NO: 1), a major capsid protein having at least 95%, at least 96%, at least 97%, at least 98%, at least 99%, or 100% sequence identity to Accession No. YP_001129378.1 (SEQ ID NO: 2), and an ORF4 protein having at least 95%, at least 96%, at least 97%, at least 98%, at least 99%, or 100% sequence identity to Accession No. YP_001129351.1 (SEQ ID NO: 3).

### Virus-Like Vesicles (VLVs)

Virus-like vesicles (VLVs) are membrane-enclosed vesicles that resemble native enveloped viruses but lack the viral capsid. VLVs are secreted alongside live virions during the lytic infection of herpesviruses including KSHV, Epstein-Barr virus (EBV), and KSHV's murine homolog, murine gammaherpesvirus 68 (MHV68). VLVs contain the complete repertoire of viral envelope glycoproteins and some tegument proteins, but lack capsid and capsid-associated proteins. While VLVs are able to fuse to and enter cells, the VLVs are replication incompetent and are unable to generate either lytic or latent infection.

Previously, VLVs were made from KSHV mutants that were defective in the small capsid protein (SCP, ORF65). See Gong et al. (2017). While the ORF65 mutations produced VLVs, residual capsid formation and DNA packaging unfortunately led to the production of some infectious virions along with the VLVs. That is, the prior art VLVs contained small amounts of infectious virions.

Thus, as disclosed herein, to produce VLVs without any virions, a KSHV mutant having a deletion of the first 60 amino acids of the major capsid protein (ORF25) was used. This “ORF25Δ60 mutant” is deficient in capsid assembly as a result of the ORF25Δ60 mutation. Particularly, the ORF25Δ60 mutant was used to infect iSLK cells. The iSLK cells harboring the ORF25Δ60 mutant genome are referred to herein as “iSLK-25Δ60 cells”.

The iSLK-25Δ60 cells and iSLK cells comprising wild-type KSHV (“iSLK-WT cells”) were induced to enter lytic replication with doxycycline and 1 mM sodium butyrate (NaB) to produce VLVs. About 4 to 5 days after induction when 90% of the cells were round and detach from the plate, the supernatant was harvested and the VLVs contained therein were purified. Specifically, the supernatant was centrifuged at 10,000 g for 10 minutes to pellet cell debris. The clarified supernatant was pelleted by ultracentrifugation at 21,000 g for 90 minutes at 4° C. Then the VLV pellets were further purified by centrifugation through 25% sucrose over a 50% sucrose cushion. Particles at the sucrose interface were pelleted by centrifugation after which the VLV pellets were resuspended in PBS.

The protein contents of the virions from the iSLK-WT cells and the VLVs from both were quantified by Bradford assay. Cryo-electron microscopy of the resuspended constituents confirmed the presence of virions in the preparations obtained from the iSLK-WT cells and the lack of virions in the samples obtained from the iSLK-25Δ60 cells. Specifically, in addition to VLVs, virions containing capsids were observed in samples obtained from iSLK-WT cells. The samples from the iSLK-25Δ60 cells, however, contained vesicles around 200 nm in diameter and lacked the presence of capsids. The VLVs produced using the ORF25Δ60 mutant are herein referred to as ΔVLVs.

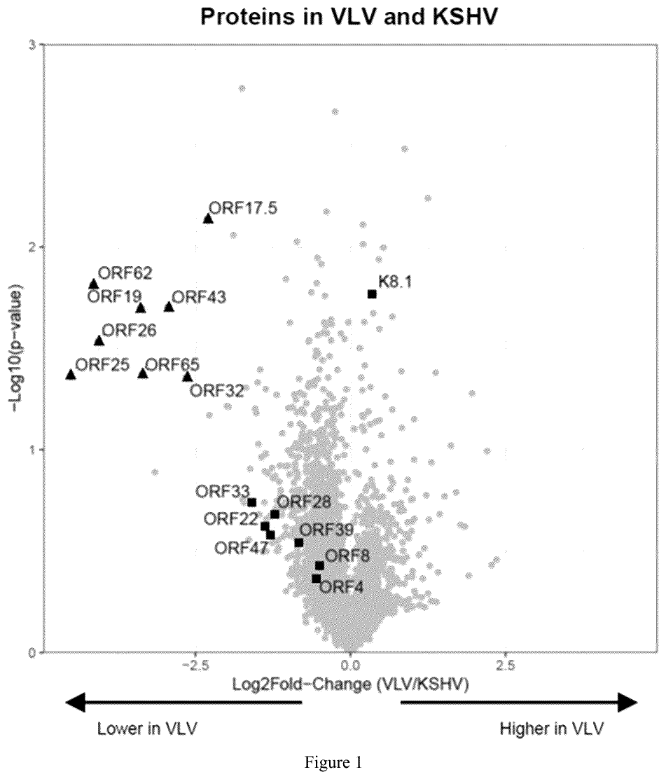

To demonstrate the presence of KSHV antigens, immunogold staining for viral glycoprotein K8.1 was performed. Both samples contained labeled VLVs and the wildtype samples also contained labeled virions. The viral protein contents were further characterize using mass spectrometry. Samples from the iSLK-25Δ60 cells contained (a) similar levels of viral envelope proteins such as ORF8 (gB) and K8.1 (FIG. 1, squares), and (b) significantly lower levels of capsid associated proteins such as ORF25 and ORF65 (FIG. 1, triangles) as compared to samples obtained from the iSLK-WT cells. Other viral proteins including tegument proteins and cellular proteins were also detected in the VLVs (Table 1 and Table 2). These results indicate that VLVs generated from KSHV mutants that are incapable of capsid formation still present a diverse array of KSHV antigens.

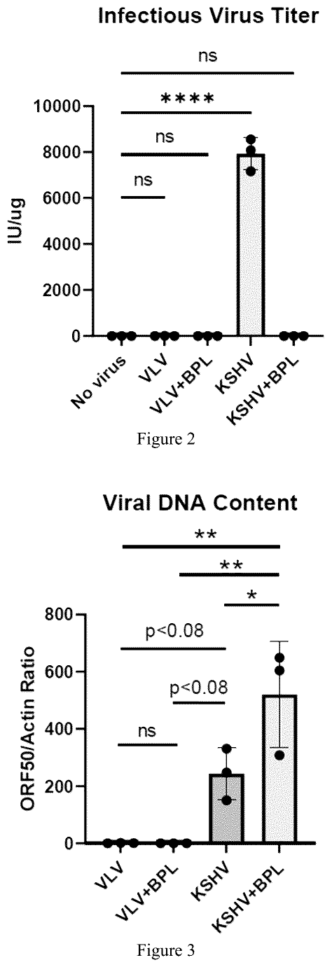

HEK293 cells were incubated with the ΔVLVs to confirm that they are not infectious. KSHV derived from BAC16 contains expression cassettes for green fluorescence protein (GFP) and hygromycin resistance in the genome. Thus, cells infected with BAC16-derived viruses will express GFP and will be resistant to hygromycin. No significant amount of GFP-expressing cells after incubation with ΔVLVs was detected using flow cytometry. That is, the levels of GFP expression were similar that of negative controls, i.e., cells incubated without KSHV and cells incubated with KSHV virions that were inactivated with beta-propiolactone (BPL) (KSHV+BPL) (FIG. 2). On the other hand, when incubated with an equal protein amount of KSHV, about 8,000 GFP-positive cells per g of protein were detected.

Hygromycin selection was also performed to confirm that the ΔVLVs do not lead to the establishment of infection. Specifically, cells were incubated with 1 μg of ΔVLVs or KSHV and monitored for 3 weeks for drug resistance. While cells continued to proliferate after incubation with KSHV, no surviving cells were found after incubation with ΔVLVs. This data confirms that VLVs prepared from KSHV mutants lacking the ability to form capsids are not infectious.

DNA from ΔVLVs and KSHV virions was isolated to assess the amount of viral DNA. ΔVLVs contained significantly less viral DNA as compared to KSHV virions, which is presumably the result of the inability of the ORF25Δ60 mutant to form a capsid around its viral genome (FIG. 3). Overall, these experiments indicate that VLVs prepared from KSHV mutants lacking the ability to form capsids present a repertoire of KSHV envelope proteins that is similar to KSHV virions, but without the risk of latent infection.

### Immune Responses to ΔVLVs

The immunogenicity of ΔVLVs was examined and compared to inactivated KSHV virions. Thus, both ΔVLVs and KSHV virions were subjected to the same chemical inactivation protocol. Specifically, KSHV virions and ΔVLVs were resuspended in 1 mL DPBS containing 100 mM sodium phosphate (Fisher Scientific #S374-500). 1 μL beta-propiolactone (BPL, Alfa Aesar, Haverhill, MA #AAB2319703) was added for a final concentration of 0.1% (v/v). This solution was inverted at room temperature overnight to inactivate virus. BPL was removed by dialysis against DPBS at 4° C. overnight using 15 kDa molecular weight cut-off TUBE-O-DIALYZER (G-Biosciences, St. Louis, MO #786618). BPL treated ΔVLVs (VLV+BPL) and KSHV (KSHV+BPL) were used for immunizations.

Antibody Immune Responses

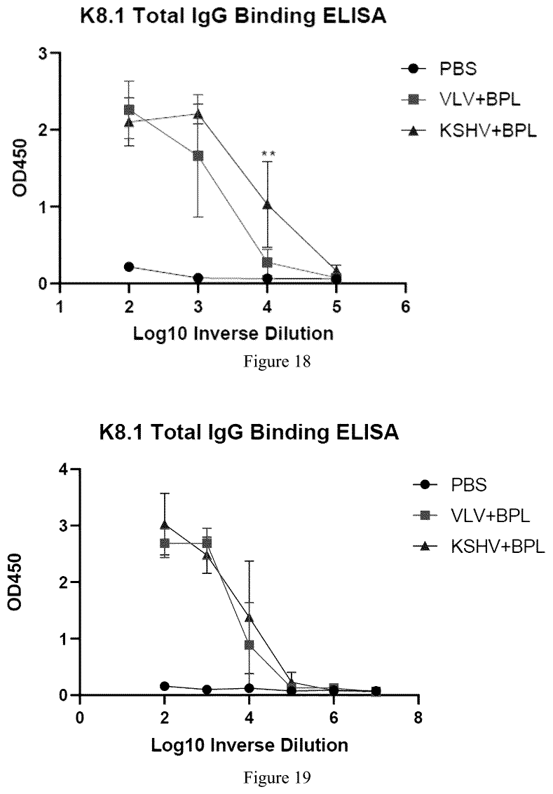

Mice were immunized intraperitoneally with 2 μg of BPL-treated VLV (VLV+BPL) or BPL-treated KSHV virions (KSHV+BPL) three times at an interval of 3 weeks. Immune sera collected at 14 days after the third immunization was assessed for KSHV-specific antibodies (FIG. 17). An enzyme-linked immunosorbent assay (ELISA) using purified K8.1 protein was performed. VLV immunization elicited a lower level of K8.1 antibodies compared to inactivated KSHV virions (FIG. 18). To identify other targets of antibody responses, a bead-based multiplexed assay that included 62 KSHV proteins was used and resulted in the identification of ORF4 and K8.1 as targets of antibodies generated by VLV immunization. Immunization of KSHV+BPL generated a broader antibody response to multiple proteins besides K8.1 and ORF4, such as tegument protein ORF38 and the small capsid protein ORF65.

Thus, ΔVLVs prepared from KSHV mutants lacking the ability to form capsids may be used as a vaccine to induce the production of antibodies against KSHV.

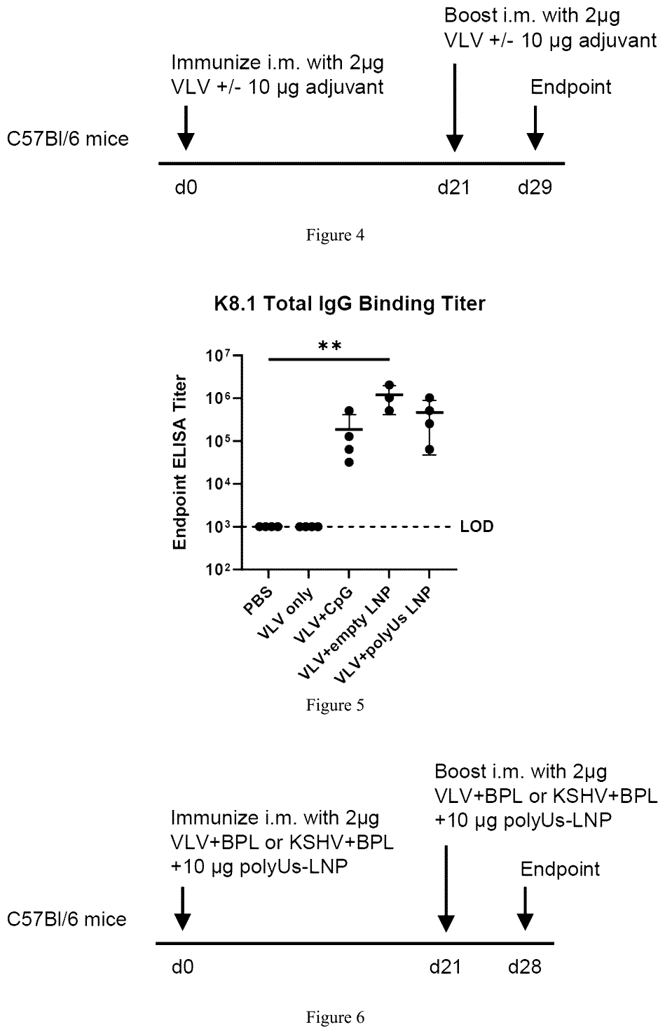

Immune responses by intramuscular immunization was also assessed. To increase immunogenicity, the ΔVLVs were co-administered with one of the following 3 types of adjuvants: (1) a CpG oligodeoxynucleotide (CpG ODN) agonist of Toll-Like Receptor 9 (TLR9), (2) empty lipid nanoparticles (LNPs), and (3) phosphorothioate-linked 21-mer polyU oligonucleotide encapsulated in lipid nanoparticles (polyUs-LNPs). Mice were immunized 2 times with 2 μg of ΔVLVs alone or in combination with 10 μg of one of the three adjuvants at a 3-week interval. Mice were immunized twice with 2 μg of ΔVLVs alone or in combination with 10 μg of one type of adjuvant at a 3-week interval (FIG. 4). The serum antibody responses at 8 days after the second immunization were then examined. All three types of adjuvants enhanced the immunogenicity of the ΔVLVs to elicit antibody responses against K8.1. Immunofluorescence assays indicate ΔVLV sera (sera from subjects immunized with ΔVLVs) contain antibodies against antigens that are expressed during the lytic viral cycle. ELISA experiments indicate that immunization with ΔVLVs alone fails to generate detectable anti-K8.1 IgG antibodies, and all three types of adjuvants enhanced the immunogenicity of ΔVLVs to elicit anti-K8.1 antibodies (FIG. 5).

In a separate intramuscular immunization, the immunogenicity of VLV+BPL was compared with KSHV+BPL adjuvanted with polyUs-LNPs (FIG. 6). Using a small panel of expression plasmids for viral envelope proteins, the VLV+BPL immune sera were shown to contain antibodies that recognized surface expression of K8.1, ORF4 and gB, but not gH/gL, gM/gN, or ORF28. ΔVLVs elicited a similar level of K8.1-binding antibodies to inactivated virions (FIG. 19).

The antibody profiles were examined using a ORFeome-wide assay. Other than the two envelope proteins, K8.1 and ORF4, VLV+BPL adjuvanted with polyUs-LNPs could also induce significant antibodies against ORF38, which were only weakly detected when ΔVLVs were administered intraperitoneally without adjuvants. The antibody responses to these three proteins were comparable between immunization of ΔVLVs and inactivated virions. ΔVLVs did not generate antibodies against the small capsid protein, ORF65, as inactivated virions did. Nevertheless, when co-delivered with adjuvants via an intramuscular route, ΔVLVs and inactivated virions exhibit similar capacities for inducing antibodies against envelope proteins.

Thus, adjuvants such as polyUs-LNPs may be used to enhance humoral immunity against KSHV resulting from immunization with ΔVLVs prepared from KSHV mutants lacking the ability to form capsids.

### Cellular Immune Responses

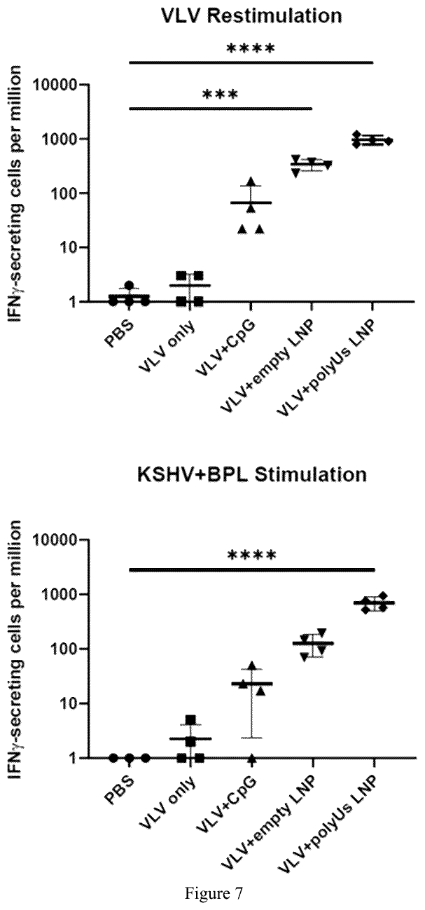

In addition to enhancing the ability of ΔVLVs to elicit antibodies against KSHV proteins, polyUs-LNPs also enhanced the ability of ΔVLVs to elicit T cell responses against KSHV at levels greater than that obtained using CpG or LNPs as an adjuvant (FIG. 7). Particularly, the incorporation of 21-mer uridine-containing single-stranded RNA (polyUs) further enhanced the ability of ΔVLVs to elicit T cell responses after intramuscular immunizations. T cell responses in splenocytes isolated from mice immunized according to the protocol shown in FIG. 4 were assayed using interferon gamma (IFNγ) enzyme-linked immunosorbent spot (ELISpot) assays. Cells were stimulated with ΔVLVs or inactivated KSHV virions and IFNγ-producing cells responding to stimulation were quantified. ΔVLVs without adjuvant did not elicit any responding T cells. However, significant numbers of IFNγ-producing cells were observed in subjects that received adjuvanted immunizations and polyUs-LNPs more potently stimulated cellular immunity than empty LNPs and CpGs (FIG. 7).

An activation induced marker (AIM) assay to examine T cell responses was also used. The AIM assay measures the activation of T cells based on the upregulation of the markers CD69 and 4-1BB for CD8 T cells and OX40 and 4-1BB for CD4 T cells. They have been used to detect T cells generated by both infection and vaccination. Splenocytes from mice immunized according to the protocol shown in FIG. 6 were stimulated with VLV+BPL or KSHV+BPL and analyzed by flow cytometry for the expression of activation markers. AIM+ CD4 and CD8 T cells were detected in subjects immunized with ΔVLVs or inactivated KSHV virions (FIG. 8). Moreover, both types of vaccines induced similar levels of AIM+ T cells in mice.

To determine which viral proteins were targets for T cell responses, a proteome-wide ELISpot assay using overlapping peptide libraries derived from viral antigens was conducted. Subjects immunized with ΔVLVs exhibited consistent T cell responses to ORF33 while subjects immunized with KSHV virions exhibited T cells responses to both ORF33 and ORF25.

These experiments indicate ΔVLVs prepared from KSHV mutants lacking the ability to form capsids can be used in combination with an adjuvant such as polyUs-LNPs be used to provide cellular immunity against KSHV.

Viral Neutralization

Immunofluorescence assays indicate that ΔVLV sera contain robust antibodies against K8.1 and ORF4 and relatively weaker antibody responses against viral fusion proteins, such as gB, gH and gL.

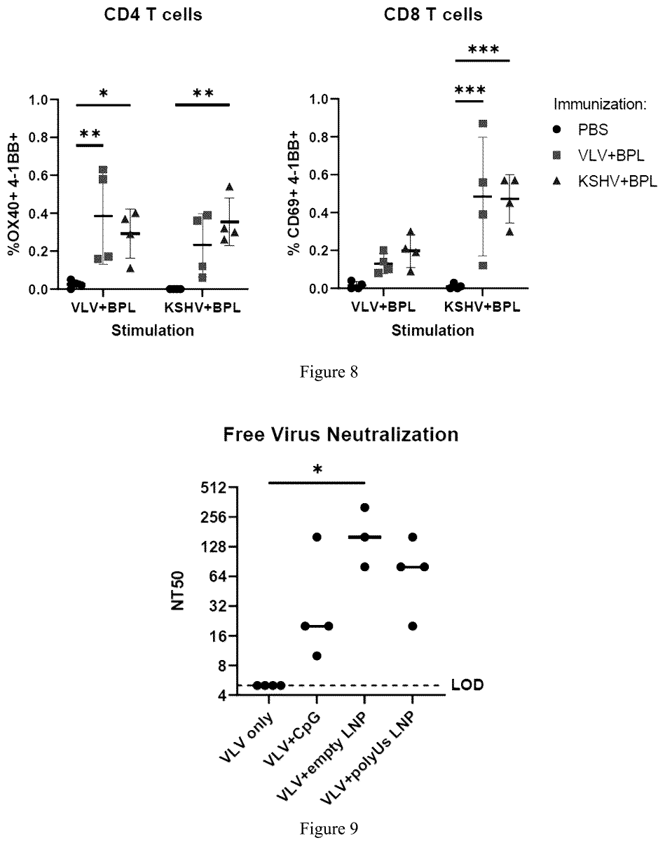

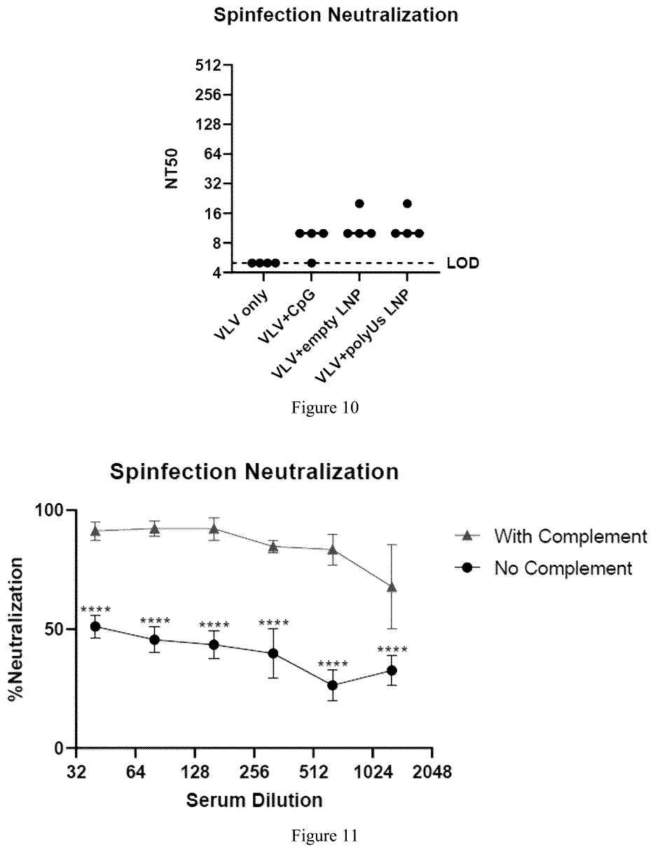

To determine whether vaccine-induced antibodies block attachment and entry, a neutralization assay based on GFP-expression in infected cells was conducted. Serial dilutions of the serum samples from mice immunized according to the protocol shown in FIG. 4 were mixed with a fixed amount of KSHV virions that lead to about 5% GFP-positive cells. The 50% neutralization titer (NT50) was determined by the last dilution of immune serum that gave more than a 50% reduction in GFP-positive cells compared to the serum samples from mock immunized mice. While neutralizing activity was undetectable when ΔVLVs alone were used for immunization, co-administration of adjuvants enabled ΔVLVs to induce significant amounts of neutralizing antibodies (FIG. 9). When spin infection was used in the neutralization assay to facilitate viral attachment by centrifugation, the NT50 values dropped but were still detectable (FIG. 10). This result is consistent with the observation that strong antibody responses were detected against viral attachment proteins while the responses to fusion proteins were much weaker.

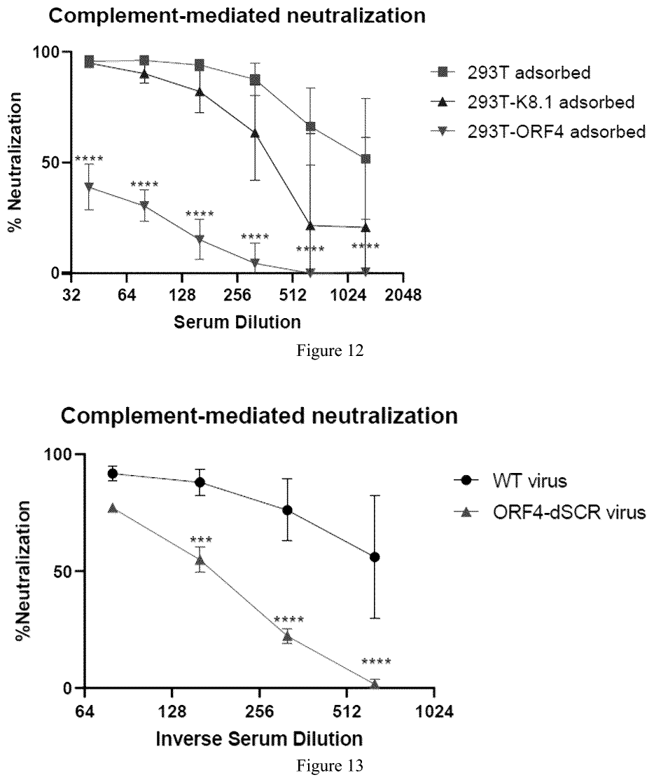

Spin infection neutralization assays were performed using sera from mice immunized with VLV+BPL according to the protocol shown in FIG. 6 and guinea pig serum (GPS) as a source of complement. Incubating KSHV virions with GPS without the immune serum did not result in any reduction in GFP-positive cells (data not shown). However, when the ΔVLV sera was included with GPS, a significant increase in neutralization was observed (FIG. 11). To determine the role of anti-ORF4 antibodies in this complement-enhanced neutralization, an adsorption procedure to deplete ORF4 antibodies by incubating the immune serum samples with ORF4-expressing cells was utilized. A similar adsorption by incubating with K8.1-expressing cells was carried out to deplete K8.1-specific antibodies. Antibody depletions were confirmed by IFA. Depletion of anti-ORF4 antibodies caused a significant loss of complement-enhanced neutralization, while depletion of K8.1 antibodies had little impact until higher serum dilutions were used (FIG. 12). These results indicate that anti-ORF4 antibodies may be used for complement-neutralization of KSHV.

To determine whether anti-ORF4 antibodies counteract the complement inhibitory function of ORF4 on viral envelope to enhance complement-mediated neutralization, a viral mutant with a deletion in the short consensus repeat (SCR) region of ORF4 was generated (“ORF4-dSCR mutant”). Specifically, the ORF4-dSCR mutant contains an ORF4 that lacks amino acid residues 24-314 as compared to the wildtype ORF4 protein (Accession No. YP_001129351.1). The ORF4-dSCR mutant was expected to be unable to inhibit complement, leading to an increased sensitivity to complement even in the absence of anti-ORF4 antibodies. However, the ORF4-dSCR mutant remained resistant to complement, indicating that KSHV virions likely do not activate the complement system in an antibody-independent manner. In the spin infection neutralization assay with ΔVLV sera, the ORF4-dSCR mutant was less susceptible to complement-enhanced neutralization compared to the WT virus (FIG. 13). This result suggests that the SCR region of ORF4 is needed for the formation of the antibody complex to activate the complement system.

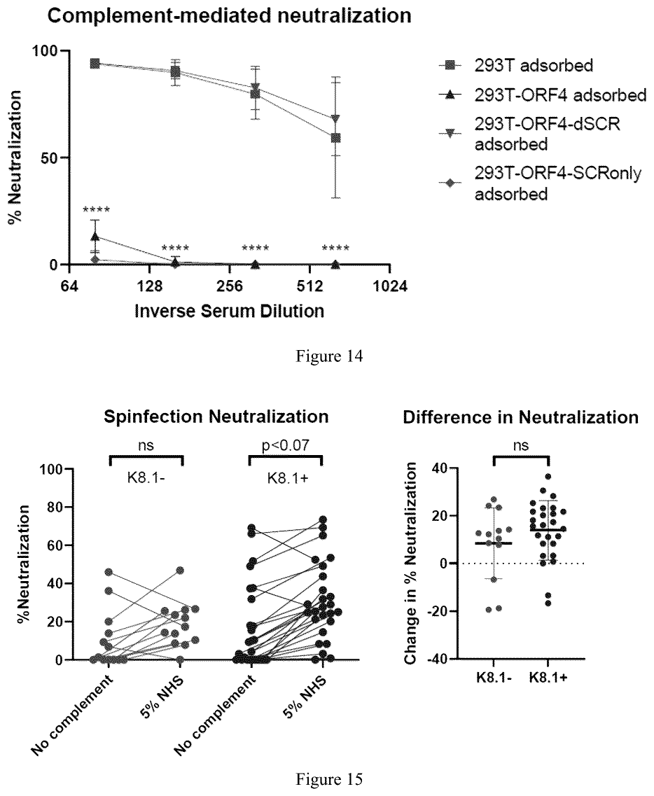

To confirm the importance of SCR-binding antibodies in complement-dependent neutralization, plasmids that express the SCR region only (ORF4-SCRonly) or the rest of ORF4 protein (ORF4-dSCR) were generated for antibody depletion. IFA indicates that anti-ORF4 antibodies of the ΔVLV sera mainly targeted the SCR region. When SCR-specific antibodies were depleted by adsorption on SCR-expressing cells, complement-enhanced neutralization was abolished, whereas the depletion of antibodies binding to the other non-SCR part of ORF4 had no impact (FIG. 14). Thus, the antibodies binding to the SCR region of ORF4 are key to recruiting and activating the complement system to facilitate neutralization of KSHV virions.

These results indicate that VLVs prepared from KSHV mutants lacking the ability to form capsids induce the production of anti-ORF4 antibodies that engage complement-mediated neutralization of virus.

### Antibodies of KSHV-Seropositive Subjects Lack Complement-Mediated Neutralization

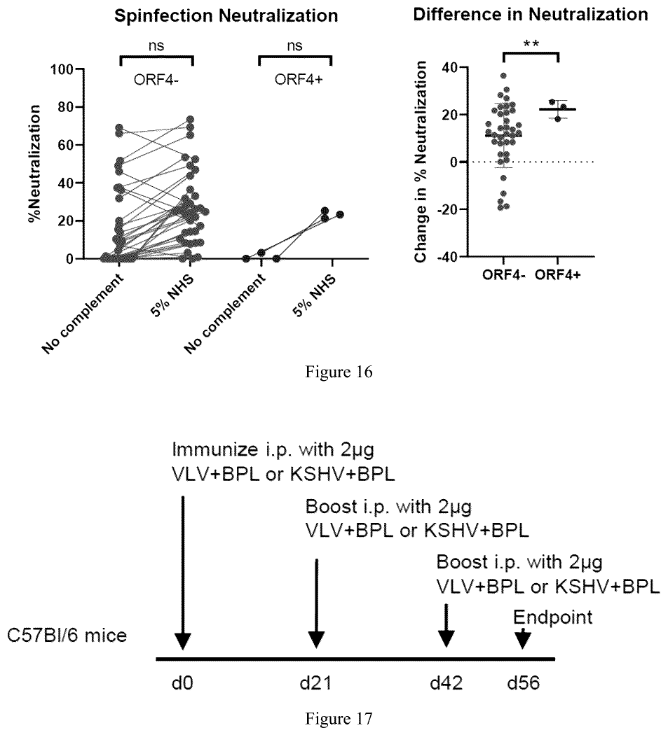

Serum samples were obtained from patients with and without KS from the multicenter AIDS cohort study (MACS). These serum samples were stratified for the presence of anti-K8.1 antibodies and anti-ORF4 antibodies by ELISA. Out of 35 KS+ patients, 25 were positive for K8.1 antibodies, but only 3 were positive for ORF4 antibodies. All 3 samples positive for ORF4 antibodies were also positive for K8.1 antibodies. Neutralization assays were performed on all 35 serum samples at a 1:100 dilution with and without human normal serum (NHS) as the source for complement. Only 3 samples reduced the percentage of GFP+ cells after spin infection by more than 50% without complement and 2 of these samples displayed a moderate increase in neutralization when complement was included. While enhanced neutralization by complement was observed for most samples, only a few achieved over 50% neutralization. When samples were separated by K8.1 or ORF4 antibody status (FIG. 15 and FIG. 16), anti-ORF4 antibody positive samples were significantly different from anti-ORF4 antibody negative samples when looking at the change in neutralization induced by adding complement (FIG. 16, right). Overall, the sera of KSHV-infected human patients in the UCLA MACS repository have little neutralizing activity and do not display significant levels of complement-enhanced neutralization.

These results indicate that infection by KSHV does not result in anti-ORF4 antibodies that result in a significant complement-mediated neutralization of virus. Therefore, subjects who are infected with KSHV may benefit from immunization with VLVs prepared from KSHV mutants lacking the ability to form capsids and/or administration of anti-ORF4 antibodies that were generated against VLVs prepared from KSHV mutants lacking the ability to form capsids.

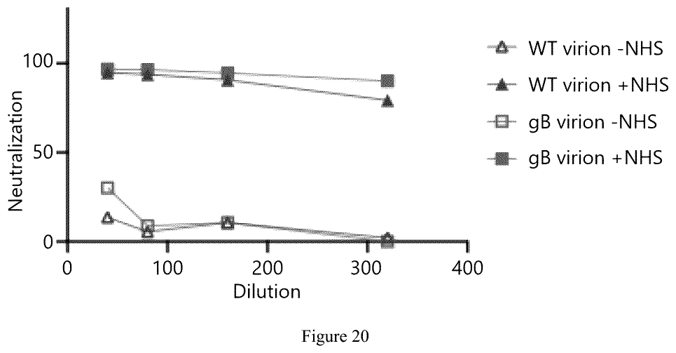

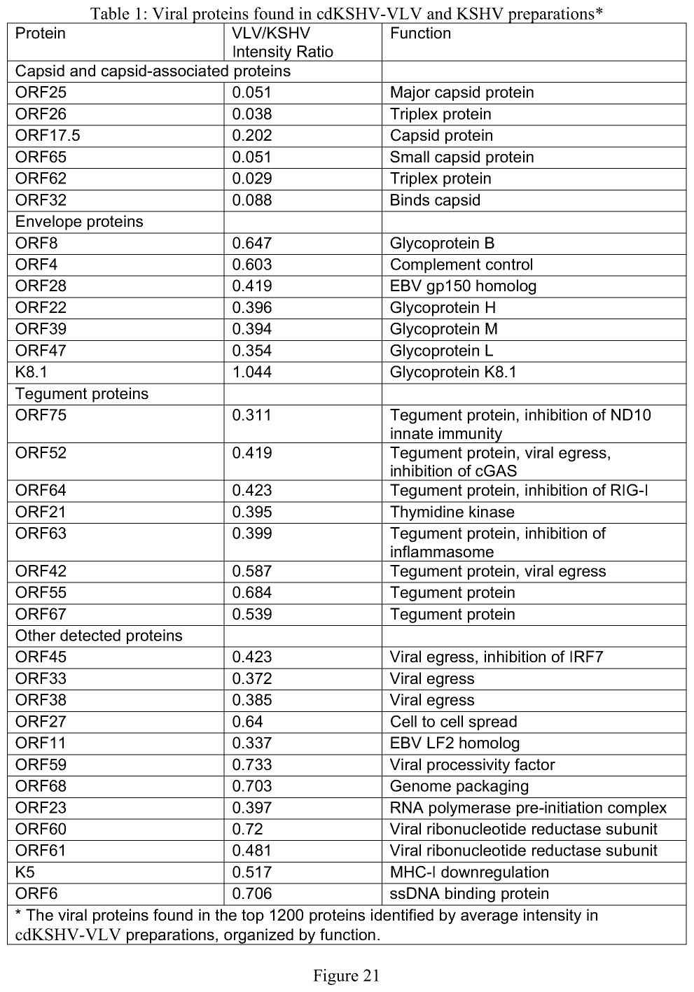

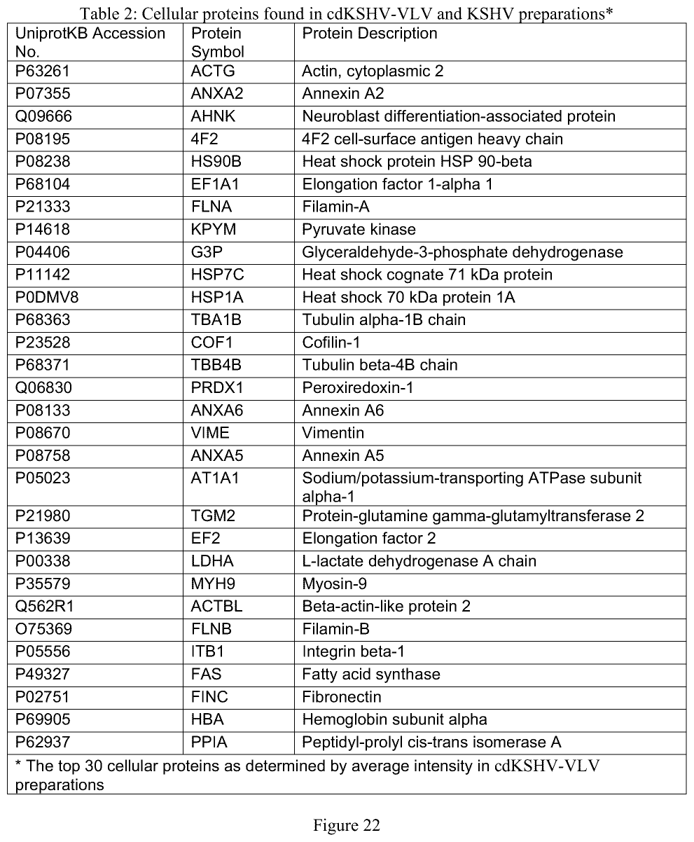

As shown in FIG. 20, immunization with mutant gB virions results in the production of antibodies that mediate complement-dependent neutralization. Specifically, mutant gB virions induce substantially comparable antibody responses as chemically inactivated wildtype virions. Particularly, in the presence of a complement source (+NHS), the induced antibodies exhibit substantially similar levels of complement-dependent neutralization of wildtype herpesvirus. Because the mutant gB virions are non-infectious virions, mutant gB virions have a better safety profile compared to chemically inactivated wildtype herpesviruses.

### VLVs

The experiments herein indicate that VLVs made from capsid deficient mutants of a given herpesvirus may be used as an alternative to whole inactivated virions (WIV) to immunize against subjects against the given herpesvirus. The capsid deficient mutants are deficient in the major capsid protein. Because the capsid deficient mutants are deficient in the major capsid protein (instead of the small capsid protein), VLVs made therefrom are free of infections virions and therefore have a better safety profile than VLVs made from capsid deficient mutants that are deficient in the small capsid protein.

Therefore, in some embodiments, the present invention is directed to virus-like vesicles made from capsid deficient mutants (deficient in the major capsid protein) of herpesviruses (viruses belonging to the Herpesviridae family), which are herein referred to as “cdHV-VLVs” and methods of making. Viruses belonging to the Herpesviridae family include those belonging to the Alphaherpesvirinae subfamily (i.e., Iltovirus, Mardivirus, Scutavirus, Simplexvirus, and Varicellovirus); Betaherpesvirinae subfamily (i.e., Cytomegalovirus, Muromegalovirus, Proboscivirus, Quwivirus, and Roseolovirus); and Gammaherpesvirinae subfamily (i.e., Bossavirus, Lymphocryptovirus, Macavirus, Manticavirus, Patagivirus, Percavirus, and Rhadinovirus). In some embodiments, the cdHV-VLVs are made from a virus selected from Herpes simplex virus-1 (HSV-1, HHV-1), Herpes simplex virus-2 (HSV-2, HHV-2), Varicella zoster virus (VZV, HHV-3), Epstein-Barr virus (EBV, HHV-4), Cytomegalovirus (CMV, HHV-5), HHV-6A, HHV-6B, HHV-7, Monkey B virus (CeHV-1), Murid gammaherpesvirus 68 (MuHV-68), and Kaposi's Sarcoma-Associated Herpesvirus (KSHV), which has been genetically modified to be capsid deficient. In some embodiments, the cdHV-VLVs are made from a virus selected from Gammaherpesvirinae subfamily, which virus which has been genetically modified to be capsid deficient. In some embodiments, the cdHV-VLVs are made from Ateline gammaherpesvirus 2, Ateline gammaherpesvirus 3, Bovine gammaherpesvirus 4, Cricetid gammaherpesvirus 2, Human gammaherpesvirus 8, Macacine gammaherpesvirus 5, Macacine gammaherpesvirus 8, Macacine gammaherpesvirus 11, Macacine gammaherpesvirus 12, Murid gammaherpesvirus 4, Murid gammaherpesvirus 7, Saimiriine gammaherpesvirus 2, that has been genetically modified to be capsid deficient.

In KSHV, the major capsid is ORF25. As used herein, “cdKSHV-VLVs” refer to VLVs made from capsid deficient mutant of KSHV. Exemplary cdKSHV-VLVs include ΔVLVs. Capsid deficient KSHV mutants are known in the art (e.g., Dai et al. (2018)) and include KSHV viruses that have been recombinantly modified to have a deletion of at least 25 of the first amino acid residues of and up to the entire ORF25 protein. Exemplary capsid deficient KSHV mutants include those having one of the following mutations: ORF25Δ30, ORF25Δ31, ORF25Δ32, ORF25Δ33, ORF25Δ34, ORF25Δ35, ORF25Δ36, ORF25Δ37, ORF25Δ38, ORF25Δ39, ORF25Δ40, ORF25Δ41, ORF25Δ42, ORF25Δ43, ORF25Δ44, ORF25Δ45, ORF25Δ46, ORF25Δ47, ORF25Δ48, ORF25Δ49, ORF25Δ50, ORF25Δ51, ORF25Δ52, ORF25Δ53, ORF25Δ54, ORF25Δ55, ORF25Δ56, ORF25Δ57, ORF25Δ58, ORF25Δ59, ORF25Δ60, ORF25Δ61, ORF25Δ62, ORF25Δ63, ORF25Δ64, and ORF25Δ65, as compared to the wildtype sequence of ORF25. Preferred capsid deficient mutants have one of the following mutations: ORF25Δ32, ORF25Δ33, ORF25Δ34, ORF25Δ35, ORF25Δ36, ORF25Δ37, ORF25Δ38, ORF25Δ39, ORF25Δ40, ORF25Δ41, ORF25Δ42, ORF25Δ43, ORF25Δ44, ORF25Δ45, ORF25Δ46, ORF25Δ47, ORF25Δ48, ORF25Δ49, ORF25Δ50, ORF25Δ51, ORF25Δ52, ORF25Δ53, ORF25Δ54, ORF25Δ55, ORF25Δ56, ORF25Δ57, ORF25Δ58, ORF25Δ59, and ORF25Δ60, as compared to the wildtype sequence of ORF25. As used herein, the notation “ORF25A” followed by a number indicates an amino acid deletion the given number of amino acid residues at the N-terminus of the given ORF25 protein when optimally aligned with the ORF25 wildtype sequence. For example, “ORF25Δ60” indicates an ORF25 protein that lacks amino acids 1-60 of wildtype ORF25. The Accession number of the wildtype ORF25 protein sequence is YP_001129378.1 (SEQ ID NO: 2). In some embodiments, excluding the deleted N-terminal end, the ORF25 protein of a capsid deficient KSHV has at least 95%, at least 96%, at least 97%, at least 98%, at least 99%, or 100% sequence identity to the wildtype ORF25 protein sequence. In some embodiments, excluding the ORF25 protein, the capsid deficient KSHV mutants have at least 95%, at least 96%, at least 97%, at least 98%, at least 99%, or 100% sequence identity to wildtype KSHV (Accession No. NC_009333.1).

Methods of making cdHV-VLVs such as cdKSHV-VLV generally comprise inducing lytic replication of a capsid deficient herpes virus in cells to obtain the VLVs as described herein. In some embodiments, the capsid deficient herpes virus is a capsid deficient KSHV mutant. In some embodiments, the capsid deficient KSHV mutant comprises a mutation selected from ORF25Δ30, ORF25Δ31, ORF25Δ32, ORF25Δ33, ORF25Δ34, ORF25Δ35, ORF25Δ36, ORF25Δ37, ORF25Δ38, ORF25Δ39, ORF25Δ40, ORF25Δ41, ORF25Δ42, ORF25Δ43, ORF25Δ44, ORF25Δ45, ORF25Δ46, ORF25Δ47, ORF25Δ48, ORF25Δ49, ORF25Δ50, ORF25Δ51, ORF25Δ52, ORF25Δ53, ORF25Δ54, ORF25Δ55, ORF25Δ56, ORF25Δ57, ORF25Δ58, ORF25Δ59, ORF25Δ60, ORF25Δ61, ORF25Δ62, ORF25Δ63, ORF25Δ64, and ORF25Δ65, as compared to the wildtype sequence of ORF25. In some embodiments, excluding the deleted N-terminal end, the ORF25 protein of a capsid deficient KSHV has at least 95%, at least 96%, at least 97%, at least 98%, at least 99%, or 100% sequence identity to the wildtype ORF25 protein sequence. In some embodiments, excluding the ORF25 protein, the capsid deficient KSHV mutants have at least 95%, at least 96%, at least 97%, at least 98%, at least 99%, or 100% sequence identity to wildtype KSHV (Accession No. NC_009333.1). In some embodiments, the cells are iSLK cells.

A cdHV-VLV for a given herpes virus may be further modified to, e.g., broaden T cell responses, by incorporating viral genes and fragments from other herpes viruses. For example, where the given herpes virus to which a cdHV-VLV is directed is KSHV (i.e., the cdHV-VLV is a cdKSHV-VLV), one or more viral genes or fragments thereof of EBV may be added to the genome of the mutant virus that is used to generate the VLVs. For example, a nucleic acid molecule encoding a tegument protein, e.g., UL16, or fragment thereof or a latency-associated viral protein, e.g., LMP-1, or a fragment thereof, may be recombinantly fused to a KSHV gene contained in the genome of the capsid deficient KSHV mutant.

### Antibodies

### Antibodies Against Non-Infectious Virions and VLVs

The experiments herein indicate that antibodies raised against non-infectious virions, e.g., mutant gB virions, and VLVs made from major capsid protein deficient mutants exhibit superior complement-mediated neutralization of the given herpesvirus from which the mutants are derived. Therefore, in some embodiments, the present invention provides methods of making complement-mediated neutralizing antibodies against herpesviruses and antibodies and sera made by said methods. The neutralizing antibodies and sera may be made by immunizing a subject with one or more gB virions and/or one or more VLVs as described herein and then obtaining the resulting sera from the subject. In some embodiments, the antibodies may be isolated and their CDR regions sequenced using methods in the art. In some embodiments, synthetic antibodies and binding fragments made using recombinant methods in the art by, e.g., by grafting the CDR regions of an antibody that was raised against a non-infectious virion or VLV described herein onto the backbone structure of an antibody or antibody fragment, nanobody, affimer, etc.

### Anti-ORF4-Antibodies

The experiments herein indicate that antibodies raised against ORF4-SCR proteins play a role in complement-mediated neutralization of KSHV. Therefore, in some embodiments, the present invention provides anti-ORF4 antibodies, compositions comprising one or more anti-ORF4 antibodies, and methods of making anti-ORF4 antibodies. Anti-ORF4 antibodies may be made by immunizing a subject with one or more ORF4-SCR agents. For example, a subject may be administered one or more ORF4-SCR proteins as freely soluble proteins or by way of VLVs or non-infectious viral particles that present one or more ORF4-SCR proteins on their surfaces. Alternatively, a subject may be administered a nucleic acid molecule that encodes an ORF4-SCR protein, which is expressed in the subject. In some embodiments, the anti-ORF4 antibodies are isolated and their CDR regions are sequenced using methods in the art. In some embodiments, synthetic antibodies and binding fragments against ORF4 are made using recombinant methods in the art by, e.g., grafting the CDR regions of an anti-ORF4 antibody that was raised against an ORF4-SCR protein onto the backbone structure of an antibody or antibody fragment, nanobody, affimer, etc.

As used herein, an “ORF4-SCR agent” refers to proteins comprising amino acid residues 24-150 of an ORF4 of a KSHV (i.e., “ORF4-SCR proteins”) and nucleic acid molecules that encode ORF4-SCR proteins. Preferred ORF4-SCR agents include ORF4-SCR proteins that comprise amino acid residues 24-314 of an ORF4 of a KSHV, and nucleic acid molecules that encode the proteins. In some embodiments, the ORF4 of a KSHV has at least 95%, at least 96%, at least 97%, at least 98%, at least 99%, or 100% sequence identity to the wildtype sequence of ORF4 (Accession No. YP_001129351.1 (SEQ ID NO: 3)).

As used herein, “antibody” refers to naturally occurring and synthetic immunoglobulin molecules and immunologically active portions thereof (i.e., molecules that contain an antigen binding site that specifically bind the molecule to which antibody is directed against). As such, the term antibody encompasses not only whole antibody molecules, but also antibody multimers and antibody fragments as well as variants (including derivatives) of antibodies, antibody multimers and antibody fragments. Examples of molecules which are described by the term “antibody” herein include: single chain Fvs (scFvs), Fab fragments, Fab′ fragments, F(ab′)₂, disulfide linked Fvs (sdFvs), Fvs, and fragments comprising or alternatively consisting of, either a VL or a VH domain. The anti-ORF4 antibodies specifically bind an ORF4-SCR protein. In some embodiments, the anti-ORF4 antibodies are raised against an ORF4-SCR protein. In some embodiments, the anti-ORF4 antibodies are raised against an ORF4-SCR protein that is presented by way of a cdKSHV-VLV or a non-infectious viral particle. In some embodiments, the antibodies are monoclonal antibodies. In some embodiments, the monoclonal antibodies are obtained from rabbit-based hybridomas.

As used herein, a compound (e.g., receptor or antibody) “specifically binds” a given target (e.g., a cdKSHV-VLV or an ORF4-SCR protein) if it reacts or associates more frequently, more rapidly, with greater duration, and/or with greater binding affinity with the given target than it does with a given alternative, and/or indiscriminate binding that gives rise to non-specific binding and/or background binding. As used herein, “non-specific binding” and “background binding” refer to an interaction that is not dependent on the presence of a specific structure (e.g., an epitope). An example of an antibody that specifically binds, e.g., an ORF4-SCR protein is an antibody that binds the ORF4-SCR protein with greater affinity, avidity, more readily, and/or with greater duration than it does to other compounds.

As used herein, “binding affinity” refers to the propensity of a compound to associate with (or alternatively dissociate from) a given target and may be expressed in terms of its dissociation constant, Kd. In some embodiments, the antibodies have a Kd of 10⁻⁵ or less, 10⁻⁶ or less, preferably 10⁻⁷ or less, more preferably 10⁻⁸ or less, even more preferably 10⁻⁹ or less, and most preferably 10⁻¹⁰ or less, to their given target. Binding affinity can be determined using methods in the art, such as equilibrium dialysis, equilibrium binding, gel filtration, immunoassays, surface plasmon resonance, and spectroscopy using experimental conditions that exemplify the conditions under which the compound and the given target may come into contact and/or interact. Dissociation constants may be used determine the binding affinity of a compound for a given target relative to a specified alternative. Alternatively, methods in the art, e.g., immunoassays, in vivo or in vitro assays for functional activity, etc., may be used to determine the binding affinity of the compound for the given target relative to the specified alternative. Thus, in some embodiments, the binding affinity of the antibody for the given target is at least 1-fold or more, preferably at least 5-fold or more, more preferably at least 10-fold or more, and most preferably at least 100-fold or more than its binding affinity for the specified alternative.

In some embodiments, ORF4-SCR agents, cdHV-VLVs (e.g., cdKSHV-VLVs), non-infectious viral particles, antibodies, and sera are substantially purified. As used herein, a “substantially purified” compound refers to a compound (or biomolecule, e.g., a VLV) that is removed from its natural environment (or the environment in which it was synthesized) and/or is at least about 60% free, preferably about 75% free, and more preferably about 90% free, and most preferably about 95-100% free from other macromolecular components or compounds with which the compound (or biomolecule) is associated with in nature or from its synthesis.

### Compositions

Compositions, including pharmaceutical compositions and vaccines, comprising, consisting essentially of, or consisting of one or more “therapeutic agents” as described herein (e.g., one or more ORF4-SCR agents, one or more anti-ORF4 antibodies, one or more cdHV-VLVs (e.g., one or more cdKSHV-VLVs), and/or one or more non-infectious viral particles) are contemplated herein.

The term “pharmaceutical composition” refers to a composition suitable for pharmaceutical use in a subject. Pharmaceutical compositions include vaccines. A pharmaceutical composition generally comprises an effective amount of one or more therapeutic agents and a diluent and/or carrier. A pharmaceutical composition generally comprises a therapeutically effective amount or an immunogenic amount, of one or more therapeutic agents and a pharmaceutically acceptable carrier. In addition to the one or more ORF4-SCR agents, pharmaceutical compositions may include one or more supplementary agents. Examples of suitable supplementary agents include viral envelope agents and latency-associated agents.

As used herein, “viral envelope glycoprotein agents” refer to viral envelope glycoproteins and fragments thereof of the given herpes virus to which the composition is directed, nucleic acid molecules encoding the viral envelope glycoproteins and fragments thereof, and antibodies against the viral envelope glycoproteins and fragments thereof. For example, for pharmaceutical composition directed to treating or inhibiting KSHV infections, exemplary viral envelope glycoprotein agents include K8.1, gB gH, gL, and fragments thereof, nucleic acid molecules encoding K8.1, gB gH, gL, and fragments thereof, and antibodies against K8.1, gB gH, gL, and fragments thereof. In some embodiments, the K8.1 glycoprotein comprises at least 95%, at least 96%, at least 97%, at least 98%, at least 99%, or 100% sequence identity to at least amino acid residues 27-194 of the wildtype K8.1 glycoprotein (Accession No. YP_001129404.1). In some embodiments, the gB glycoprotein comprises at least 95%, at least 96%, at least 97%, at least 98%, at least 99%, or 100% sequence identity to at least amino acid residues 27-194 of the wildtype gB glycoprotein (Accession No. YP_001129354.1). In some embodiments, the gH glycoprotein comprises at least 95%, at least 96%, at least 97%, at least 98%, at least 99%, or 100% sequence identity to at least amino acid residues 27-194 of the wildtype gH glycoprotein (Accession No. YP_001129375.1). In some embodiments, the gL glycoprotein comprises at least 95%, at least 96%, at least 97%, at least 98%, at least 99%, or 100% sequence identity to at least amino acid residues 27-194 of the wildtype gL glycoprotein (Accession No. YP_001129399.1).

As used herein, “latency-associated viral agents” refer to latency-associated viral proteins and fragments thereof of the given herpes virus to which the composition is directed, nucleic acid molecules encoding the latency-associated viral proteins and fragments thereof, and antibodies against the latency-associated viral proteins and fragments thereof. For example, for pharmaceutical composition directed to treating or inhibiting KSHV infections, latency-associated viral agents refer to a latency-associated nuclear antigen (LANA) and fragments thereof, nucleic acid molecules encoding the LANA and fragments thereof, and antibodies against the LANA and fragments thereof. In some embodiments, the LANA comprises at least 95%, at least 96%, at least 97%, at least 98%, at least 99%, or 100% sequence identity to at least amino acid residues 27-194 of wildtype LANA (Accession No. YP_001129431.1).

As used herein, an “effective amount” refers to a dosage or amount sufficient to produce a desired result. The desired result may comprise an objective or subjective change as compared to a control in, for example, in vitro assays, and other laboratory experiments. As used herein, a “therapeutically effective amount” refers to an amount that may be used to treat, prevent, or inhibit a given disease or condition in a subject as compared to a control, such as a placebo. Again, the skilled artisan will appreciate that certain factors may influence the amount required to effectively treat a subject, including the degree of the condition or symptom to be treated, previous treatments, the general health and age of the subject, and the like. Nevertheless, effective amounts and therapeutically effective amounts may be readily determined by methods in the art.

The one or more therapeutic agents may be administered, preferably in the form of pharmaceutical compositions, to a subject. Preferably the subject is mammalian, more preferably, the subject is human. Preferred pharmaceutical compositions are those comprising one or more cdKSHV-VLVs or one or more non-infectious viral particles in a therapeutically effective amount or an immunogenic amount, and a pharmaceutically acceptable vehicle. It should be noted that treatment of a subject with a therapeutically effective amount or an immunogenic amount may be administered as a single dose or as a series of several doses. The dosages used for treatment may increase or decrease over the course of a given treatment. Optimal dosages for a given set of conditions may be ascertained by those skilled in the art using dosage-determination tests and/or diagnostic assays in the art. Dosage-determination tests and/or diagnostic assays may be used to monitor and adjust dosages during the course of treatment.

Vaccines provide a protective immune response when administered to a subject. In some embodiments, a “vaccine”, is a pharmaceutical composition that comprises an immunogenic amount of at least one ORF4-SCR agent, at least one cdHV-VLV (e.g., cdKSHV-VLV) or at least one non-infectious viral particle and provides a protective immune response when administered to a subject. The protective immune response may be complete or partial, e.g., a reduction in symptoms as compared with an unvaccinated subject. As used herein, an “immunogenic amount” is an amount that is sufficient to elicit an immune response in a subject and depends on a variety of factors such as the immunogenicity of the given agent, the degree of the given disease or infection, the manner of administration, the general state of health of the subject, and the like.

The compositions as contemplated herein may include an adjuvant. As used herein, an “adjuvant” refers to any substance which, when administered in conjunction with (e.g., before, during, or after) a pharmaceutically active agent, such as an ORF4-SCR agent, a cdHV-VLV (e.g., a cdKSHV-VLV), or a non-infectious viral particle, aids the pharmaceutically active agent in its mechanism of action. Thus, an adjuvant in a vaccine is a substance that aids the ORF4-SCR agent, the cdHV-VLV, or the non-infectious viral particle, in eliciting an immune response. Exemplary adjuvants include incomplete Freund's adjuvant, alum, aluminum phosphate, aluminum hydroxide, N-acetyl-muramyl-L-threonyl-D-isoglutamine (thr-MDP), N-acetyl-nor-muramyl-L-alanyl-D-isoglutamine (CGP 11637, nor-MDP), N-acetylmuramyl-Lalanyl-D-isoglutaminyl-L-alanine-2-(1′-2′-dipa-lmitoyl-sn-glycero-3-hydroxyphosphoryloxy)-ethylamine (CGP 19835A, MTP-PE), and RIBI, which comprise three components extracted from bacteria, monophosphoryl lipid A, trehalose dimycolate and cell wall skeleton (NPL+TDM+CWS) in a 2% squalene/Tween 80 emulsion. Preferred adjuvants are those that trigger innate immune responses by signaling through toll-like receptors (TLRs). Particularly preferred adjuvants are CpG oligonucleotide (CpG ODN) agonists of TLR9, empty lipid nanoparticles (LNPs), and polyU oligonucleotides encapsulated by lipid nanoparticles (polyUs-LNPs). CpG oligonucleotides that are TLR9 agonists are known in the art. See, e.g., Bauer et al. (2001). PolyU oligonucleotide are known in the art. See, e.g., Diebold et al. (2006). PolyU oligonucleotides are preferably about 10-25 bases long, more preferably 20-, 21-, or 22-bases long. PolyU oligonucleotides preferably have phosphorothioate linkages. Lipid nanoparticles known in the art may be used to encapsulate phosphothioate-linked polyuridlyic acid. See, e.g., WO2017004143.

The experiments herein show that polyUs-LNPs enhances the humoral and cellular immune responses against immunogens, e.g., inactivated whole wild-type or mutant KSHV, cdKSHV-VLVs, KSHV proteins (e.g., ORF4 and glycoproteins K8.1, gB, gH, and gL). Therefore, compositions according to the present invention include: compositions comprising one or more cdKSHV-VLVs and an adjuvant (e.g., polyUs-LNPs), compositions comprising one or more one or more ORF4-SCR proteins (or a nucleic acid molecule encoding the one or more ORF4-SCR proteins) and an adjuvant, (e.g., polyUs-LNPs), and compositions comprising inactivated whole wild-type or mutant KSHV and polyUs-LNPs.

Pharmaceutical compositions may include one or more of the following: a pharmaceutically acceptable vehicle, pH buffered solutions, adjuvants (e.g., preservatives, wetting agents, emulsifying agents, and dispersing agents), liposomal formulations, nanoparticles, dispersions, suspensions, or emulsions, as well as sterile powders for reconstitution into sterile injectable solutions or dispersions. The compositions and formulations may be optimized for increased stability and efficacy using methods in the art. See, e.g., Carra et al., (2007) Vaccine 25:4149-4158.

As used herein, a “pharmaceutically acceptable vehicle” or “pharmaceutically acceptable carrier” are used interchangeably and refer to solvents, dispersion media, coatings, antibacterial and antifungal agents, isotonic and absorption delaying agents, and the like, that are compatible with pharmaceutical administration and comply with the applicable standards and regulations, e.g., the pharmacopeial standards set forth in the United States Pharmacopeia and the National Formulary (USP-NF) book, for pharmaceutical administration. Thus, for example, unsterile water is excluded as a pharmaceutically acceptable carrier for, at least, intravenous administration. Pharmaceutically acceptable vehicles include those known in the art. See, e.g., Remington: The Science and Practice of Pharmacy 20th ed (2000) Lippincott Williams & Wilkins, Baltimore, MD.

The pharmaceutical compositions may be provided in dosage unit forms. As used herein, a “dosage unit form” refers to physically discrete units suited as unitary dosages for the subject to be treated; each unit containing a predetermined quantity of the given therapeutic agent calculated to produce the desired therapeutic effect in association with the required pharmaceutically acceptable carrier. The specification for the dosage unit forms of the invention are dictated by and directly dependent on the unique characteristics of the given therapeutic agent and desired therapeutic effect to be achieved, and the limitations inherent in the art of compounding such an active compound for the treatment of individuals.

### Treatment Methods

One or more capsid deficient herpes viruses (e.g., capsid deficient KSHV), one or more cdHV-VLVs (e.g., cdKSHV-VLVs), one or more non-infectious viral particles, one or more ORF4-SCR agents, and/or one or more one or more anti-ORF4 antibodies may used to inhibit and/or treat an infection by a herpes virus in subjects. For example, in some embodiments, one or more capsid deficient herpes viruses (e.g., capsid deficient KSHV), one or more cdHV-VLVs (e.g., cdKSHV-VLVs), one or more non-infectious viral particles, one or more ORF4-SCR agents, and/or one or more one or more anti-ORF4 antibodies is administered to a subject as a prophylactic against infection by a herpes virus. In some embodiments, one or more capsid deficient herpes viruses (e.g., capsid deficient KSHV), one or more cdHV-VLVs (e.g., cdKSHV-VLVs), one or more non-infectious viral particles, one or more ORF4-SCR agents, and/or one or more one or more anti-ORF4 antibodies is administered to a subject suffering from an infection by a herpes virus, whereby the administration induces or enhances complement-mediated neutralization of the herpes virus in the subject. In some embodiments, one or more capsid deficient herpes viruses (e.g., capsid deficient KSHV), one or more cdHV-VLVs (e.g., cdKSHV-VLVs), one or more non-infectious viral particles, one or more ORF4-SCR agents, and/or one or more one or more anti-ORF4 antibodies is administered to a subject to inhibit or treat a disease, e.g., a cancer such as Kaposi's Sarcoma (KS) or primary effusion lymphoma, caused by a herpes virus. In some embodiments, the herpes virus is KSHV. In some embodiments, the one or more capsid deficient herpes viruses (e.g., capsid deficient KSHV), one or more cdHV-VLVs (e.g., cdKSHV-VLVs), one or more non-infectious viral particles, one or more ORF4-SCR agents, and/or one or more one or more anti-ORF4 antibodies are administered with an adjuvant such as polyUs-LNPs. In some embodiments, at least two different agents selected from capsid deficient herpes viruses (e.g., capsid deficient KSHV), cdHV-VLVs (e.g., cdKSHV-VLVs), non-infectious viral particles, ORF4-SCR agents, and anti-ORF4 antibodies are co-administered to a subject. For example, in some embodiments, (a) one or more cdKSHV-VLVs or one or more non-infectious viral particles, and (b) one or more anti-ORF4 antibodies are administered to a subject. As another example, two different cdHV-VLV preparations may be co-administered to a subject.

As used herein, “co-administration” refers to the administration of at least two different agents, i.e., a first agent and a second agent to a subject. In some embodiments, the co-administration is concurrent. In embodiments involving concurrent co-administration, the agents may be administered as a single composition, e.g., an admixture, or as two separate compositions. In some embodiments, the first agent is administered before and/or after the administration of the second agent. Where the co-administration is sequential, the administration of the first and second agents may be separated by a period of time, e.g., minutes, hours, or days. Those of skill in the art understand that the formulations and/or routes of administration of the various agents or therapies used may vary. The appropriate dosage for co-administration can be readily determined by one skilled in the art. In some embodiments, when two or more agents are co-administered, the respective agents are administered at lower dosages than appropriate for their administration alone.

Toxicity and therapeutic efficacy of therapeutic agents according to the instant invention and compositions thereof can be determined using cell cultures and/or experimental animals and pharmaceutical procedures in the art. For example, one may determine the lethal dose, LC₅₀ (the dose expressed as concentration×exposure time that is lethal to 50% of the population) or the LD₅₀ (the dose lethal to 50% of the population), and the ED₅₀ (the dose therapeutically effective in 50% of the population) by methods in the art. The dose ratio between toxic and therapeutic effects is the therapeutic index and it can be expressed as the ratio LD₅₀/ED₅₀. Therapeutic agents that exhibit large therapeutic indices are preferred. While therapeutic agents that result in toxic side-effects may be used, care should be taken to design a delivery system that targets such compounds to the site of treatment to minimize potential damage to uninfected cells and, thereby, reduce side-effects.

The data obtained from the cell culture assays and animal studies can be used in formulating a range of dosages for use in humans. Preferred dosages provide a range of circulating concentrations that include the ED₅₀ with little or no toxicity. The dosage may vary depending upon the dosage form employed and the route of administration utilized. Therapeutically effective amounts and dosages of one or more therapeutic agents can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography. Additionally, a dosage suitable for a given subject can be determined by an attending physician or qualified medical practitioner, based on various clinical factors.

### Kits

In some embodiments, the present invention provides kits comprising one or more capsid deficient herpes viruses (e.g., capsid deficient KSHV), one or more cdHV-VLVs (e.g., cdKSHV-VLVs), one or more non-infectious viral particles, one or more ORF4-SCR agents, and/or one or more one or more anti-ORF4 antibodies, optionally in a composition or in combination with one or more supplementary agents, packaged together with one or more reagents or drug delivery devices. In some embodiments, the kits comprise one or more capsid deficient herpes viruses (e.g., capsid deficient KSHV), one or more cdHV-VLVs (e.g., cdKSHV-VLVs), one or more non-infectious viral particles, one or more ORF4-SCR agents, and/or one or more one or more anti-ORF4 antibodies, optionally in one or more unit dosage forms, packaged together as a pack and/or in drug delivery device, e.g., a pre-filled syringe. In some embodiments, the kits comprise at least two different agents selected from capsid deficient herpes viruses (e.g., capsid deficient KSHV), cdHV-VLVs (e.g., cdKSHV-VLVs), one or more non-infectious viral particles ORF4-SCR agents, and anti-ORF4 antibodies packaged together. As another example, two different antibodies against ORF4 may be packaged together.

In some embodiments, the kits include a carrier, package, or container that may be compartmentalized to receive one or more containers, such as vials, tubes, and the like. In some embodiments, the kits optionally include an identifying description or label or instructions relating to its use. In some embodiments, the kits include information prescribed by a governmental agency that regulates the manufacture, use, or sale of compounds and compositions as contemplated herein.

The following examples are intended to illustrate but not to limit the invention.

### EXAMPLES

### Plasmids and Cell Lines

KSHV glycoprotein sequences were amplified from KSHV BAC16 derived from rKSHV.219 (a gift from Jae Jung). Sequences were cloned into the pCAG-GFPd2 overexpression vector (a gift from Connie Cepko, Addgene, Watertown, MA #14760) using the EcoRI and BglII cut sites to replace the GFP coding region or into the pcDNA3-OVA vector (a gift from Sandra Diebold and Martin Zenke, Addgene #64599) replacing the OVA coding region. SARS-CoV-2 Spike was expressed from pCMV14-3X-FLAG-SARS-CoV-2 S (a gift from Zhaohui Qian, Addgene #145780) as a negative control. K8.1 and ORF4 were cloned with FLAG tags on the extracellular domain right after the signal sequence (after amino acid 28 for K8.1 and after amino acid 20 for ORF4). ORF4 without the SCR region was cloned by removing amino acids 24-314 from the original protein sequence. The SCRonly version of ORF4 was cloned by removing amino acids 315-519 and connecting the SCR region to the transmembrane domain of ORF4 with a GGGGS linker. gB was cloned with a FLAG tag on the C terminal end. C-terminal FLAG-tagged gL was connected to C terminal HA-tagged gH with a P2A sequence for stoichiometric co-expression and proper association of the gH/gL complex. Signal peptides were determined using SignalP version 4.1 and transmembrane regions were determined using TMHMM-2.0. Cloning primers were synthesized by Integrated DNA Technologies (IDT, Coralville, IA) and plasmids were confirmed via Sanger sequencing by Laragen, Inc (Culver City, CA).

293T and HEK293 cells were cultured in Dulbecco's Modification of Eagle's Medium (DMEM) (Corning, Corning, NY #10017CV) supplemented with 10% fetal bovine serum (FBS) (Corning #35010CV) and 1× Penicillin/Streptomycin (Corning #35010CV). iSLK cells containing latent KSHV were cultured in DMEM supplemented with 10% FBS and 1× Penicillin/Streptomycin with 1 μg/mL puromycin, 250 μg/mL G418, and 1200 μg/mL hygromycin (all from Invivogen, San Diego, CA #ant-pr-1, ant-gn-2, and ant-hg-5) for selection. iSLK-WT cells harboring BAC-16 that produced GFP-expressing KSHV were a gift from Jae Jung. iSLK-25Δ60 cells were generated using methods in the art.

The ORF4-dSCR mutant was generated with en passant mutagenesis to replace amino acids 24-314 of ORF4 with the HA tag using GS1783 E. coli using methods in the art. BAC DNA containing the ORF4-dSCR mutant genome was transfected into 293T cells using Lipofectamine (Thermo Fisher Scientific, Waltham, MA #11668027). 293T stably harboring the viral genome were selected with 100 μg hygromycin. 293T cells were cocultured with iSLK cells in the presence of 20 ng/mL 12-O-tetradecanoyl-phorbol-13-acetate (Fisher Scientific, Waltham, MA #AAJ63916MCR) and 1 mM sodium butyrate (Fisher Scientific #AC263191000) to induce infection of iSLK cells. Infected iSLK cells were selected with iSLK cell media described above to establish “iSLK-ORF4-dSCR cells”.

### Virus Production, Isolation, and Inactivation

Wildtype KSHV, ORF4-dSCR, and VLVs were produced by reactivating the given latent virus in their respective iSLK cells by treating the cells with DMEM containing 10% FBS, 1× Penicillin/Streptomycin, 5 μg/mL doxycycline, and 1 mM sodium butyrate. Supernatants were harvested after 4-5 days of reactivation, when over 90% of cells exhibited cytopathic effect.

For virus used for neutralization assays, supernatants collected from 4-6 10 cm dishes of reactivated iSLK cells were clarified at 2000 g for 10 minutes, and the virus was concentrated at 21000 g for 90 minutes. Pellets were washed gently with serum-free DMEM before resuspension in serum-free DMEM. The virus suspension was clarified to remove debris by centrifugation at 7000 rpm for 3 minutes in a microcentrifuge.

For VLVs and virions used for immunization studies and T cell stimulation, supernatants collected from 20-30 15 cm dishes of reactivated iSLK cells were clarified at 8000 g for 10 minutes. Virions were pelleted by ultracentrifugation at 80,000 g for 1 hour in sterile ultracentrifuge tubes (Beckman Coulter, Indianapolis, IN #C14292), and the pellets were incubated in Dulbecco's phosphate-buffered saline (DPBS) (Thermo Fisher Scientific #14190250) overnight. The resuspended viral pellets were then loaded on top of a sucrose cushion consisting of 25% sucrose in DPBS over 50% sucrose in DPBS and centrifuged at 100,000 g for 1 hour in sterile ultracentrifuge tubes (Beckman Coulter #C14297). The band at the sucrose interface was collected, diluted in DPBS, and centrifuged at 80,000 g for 1 hour. The final pellet was incubated in DPBS overnight before resuspension. Preparations were separated into single-use aliquots immediately after resuspension and stored at −80° C. Viral protein concentration was determined by standard Bradford assay with bovine serum albumin (BSA) as standards (Fisher Scientific #BP9703100).

For chemical inactivation, KSHV or VLV were resuspended in 1 mL DPBS containing 100 mM sodium phosphate (Fisher Scientific #S374-500). 1 μL beta-propiolactone (Alfa Aesar, Haverhill, MA #AAB2319703) was added for a final concentration of 0.1% (v/v). This solution was inverted at room temperature overnight to inactivate virus. BPL was removed by dialysis against DPBS at 4° C. overnight using 15 kDa molecular weight cut-off TUBE-O-DIALYZER (G-Biosciences, St. Louis, MO #786618). Dialyzed preparations were collected, separated into single-use aliquots, and stored at −80° C.

### Non-Infectious Viral Particles

Non-infectious viral particles were made using methods in the art to introduce mutations in glycoprotein B, gB, of wildtype KSHV. Particularly, the mutant gB virions were made using methods in the art to have a mutation in their gB proteins at amino acid position 470 and/or amino acid position 471 when optimally aligned with the wildtype sequence (Accession No. YP_001129354.1). Specifically, methods in the art were used to substitute the amino acid at position 470 or 471 of the given gB protein when optimally aligned with the wildtype sequence with proline (P). Because the amino acid residues at positions 470 and 471 of the wildtype sequence are aspartic acid (D) and glycine (G), respectively, the mutant gB virions are referred to herein as gB-D470P and gB-G471P, respectively. The infectivity of the mutant gB virions was essentially abolished.

Chemically inactivated KSHV WT virions or gB mutant virions were used to immunize mice. Immunization with the chemically inactivated KSHV WT virions and immunization with the mutant gB virions induced comparable antibody responses. After 4 immunizations, sera was collected and used for neutralization assays using gB-D470P as representative of the mutant gB virions.

Neutralization assays were performed by infection of 10,000 HEK293 cells seeded overnight in a 96-well plate. The amount of virus used was calculated to give about 5% GFP+ cells for samples without neutralization. The GFP+ cells were about 20%. Thus, the titer was about 2,000 infectious units. Immune serum samples collected from mice injected with cell lysates, chemically inactivated WT virions, or gB mutant virions were diluted in DMEM containing 2% heat-inactivated normal mouse serum. For complement-dependent neutralization, KSHV was diluted with DMEM containing 10% normal human serum (Complement Technology Inc., #NHS). Diluted serum was mixed with diluted virus at a 1:1 ratio and incubated at 37° C. for 1 hour. 50 μL of the serum/virus mixture was used to infect cells via spin infection. The plate was spun at 400 g for 20 minutes before a 2-hour incubation. After the 2-hour incubation, the inoculum was removed, replaced with growth medium, and incubated for an additional 2 days. Infection was measured by flow cytometry for GFP+ cells. Neutralization was calculated as (% GFP_{cell lysate}−% GFPᵥᵢᵣᵢₒₙₛ)/% GFP_{cell lysates}*100.

### Cryo-Electron Microscopy

2.5 μL aliquots of VLV and virion preparations were applied to glow-discharged Quantifoil 200 mesh Cu R2/1 grids (Electron Microscopy Sciences, Hatfield, PA #Q250CR1). Grids were plunge-frozen in liquid ethane using an FEI Vitrobot Mark IV at 22° C. and 100% humidity. Images were acquired at 25,000× and 50,000× on an FEI Tecnai TF20 equipped with a 4 k×4 k TVIPS F415MP CCD detector.

### Immunogold Staining

2.5 μL aliquots of VLV or virion preparations were incubated on glow-discharged homemade 200 mesh formvar/carbon-coated copper grids for 5 minutes at room temperature. After sample incubation, the grids were passed through two drops of blocking buffer (PBS+0.4% BSA, filtered) on Parafilm and floated on a third drop for 30 min in a homemade moisture chamber. Excess blocking buffer was removed by lightly blotting with filter paper prior to incubation with primary antibody against K8.1 (Santa Cruz Biotechnology, Dallas, TX #sc-65446) diluted 1:50 in blocking buffer for 1 hour. Negative controls substituted primary anti-K8.1 with anti-FLAG M2 (Sigma-Aldrich, St. Louis, MO #F1804) or blocking buffer. After primary antibody incubation, the grids were washed by passing through two drops of blocking buffer and floating on a third drop for 10 minutes. The washed grids were lightly blotted before incubation with 6 nm gold-conjugated Fabs of goat anti-mouse IgG (Electron Microscopy Sciences #25374) diluted 1:20 in blocking buffer for 1 hour. The grids were washed with three drops of PBS and kept floating on a drop of PBS for at most 1 hour until ready for negative staining. For negative stain, grids were washed with three drops of distilled water and stained with 2% uranyl acetate (Electron Microscopy Sciences #22400-2) for 1 minute. Particles that were greater than 100 nm in diameter and had five or more gold beads were considered positively labeled.

### Mass Spectrometry

20 μL of each VLV or KSHV preparation was diluted with 80 μL of a master mix consisting of 43 μL HPLC water, 25 μL 8M urea, 10 μL 100 mM ammonium bicarbonate, and 1 μL 100 mM Dithiothreitol (DTT). Samples were then incubated at 60° C. to reduce disulfide linkages for 30 minutes. Iodoacetamide (IAA) was then added to a 10 mM final concentration to alkylate free cysteines and lysates were incubated in the dark at room temperature for 30 minutes. Lysates were next digested with Trypsin Gold (Promega Corporation, Madison, WI #V5280). 0.4 μg of trypsin was added to each sample, and lysates were then incubated for 16 hours at 37° C. while being vortexed at 1000 rpm. Trypsin activity was quenched by adding 10% v/v trifluoroacetic acid (TFA) to a final concentration of 0.1% TFA. Samples were then desalted on a C18 minispin column (The Nest Group, Inc., Ipswich, MA #HUM S18V) per the manufacturer's protocol. Samples were eluted from these columns with 200 μL 40% acetonitrile (ACN)/0.1% TFA. Samples were dried by vacuum centrifugation and stored at −80° C. until analysis.

All samples were analyzed on an Orbitrap Eclipse mass spectrometry system equipped with an Easy nLC 1200 ultra-high pressure liquid chromatography system (Thermo Fisher Scientific) interfaced via a Nanospray Flex nanoelectrospray source. Immediately prior to analysis, lyophilized samples were resuspended in 0.1% formic acid (FA). Samples were injected on a C18 reverse phase column (30 cm×75 m (ID)) packed with ReprosilPur 1.9 μm particles. Mobile phase A consisted of 0.1% FA, and mobile phase B consisted of 0.1% FA/80% ACN. Peptides were separated by an organic gradient from 5% to 35% mobile phase B over 120 minutes followed by an increase to 100% B over 10 minutes at a flow rate of 300 nL/minute. Analytical columns were equilibrated with 3 μL of mobile phase A. To build a spectral library individual samples were analyzed by a data-dependent acquisition (DDA) method. DDA data was collected by acquiring a full scan over a m/z range of 375-1025 in the Orbitrap at 120,000 resolution resolving power (@200 m/z) with a normalized AGC target of 100%, an RF lens setting of 30%, and an instrument-controlled ion injection time. Dynamic exclusion was set to 30 seconds, with a 10-ppm exclusion width setting. Peptides with charge states 2-6 were selected for MS/MS interrogation using higher energy collisional dissociation (HCD) with a normalized HCD collision energy of 28%, with three seconds of MS/MS scans per cycle. All individual samples were analyzed by a data-independent acquisition (DIA) method. DIA was performed on all individual samples. An MS scan was performed at 60,000 resolution (@200 m/z) over a scan range of 390-1010 m/z, an instrument controlled AGC target, an RF lens setting of 30%, and an instrument controlled maximum injection time, followed by DIA scans using 8 m/z isolation windows over 400-1000 m/z at a normalized HCD collision energy of 28%.

Spectral libraries were built with Spectronaut factory settings from DDA pools and from DDA runs from a previous SARS-CoV-2 study. Individual samples run with DIA methods were then analyzed against the before mentioned library with Spectronaut using methods in the art. False discovery rates were estimated using a decoy database strategy. All data were filtered to achieve a false discovery rate of 0.01 for peptide-spectrum matches, peptide identifications, and protein identifications. Search parameters included a fixed modification for carbamidomethyl cysteine and variable modifications for N-terminal protein acetylation and methionine oxidation. All other search parameters were defaults for the respective algorithms. Analysis of protein expression was conducted utilizing the MSstats statistical package in R. Output data from Spectronaut was annotated based on the human reference (SwissProt human reviewed sequences downloaded on Oct. 10, 2019) and the Human herpesvirus-8 (HHV8) BAC16 strain (sequences were extracted from NCBI GenBank Accession: GQ994935.1 on 03/15/2022). Technical and biological replicates were integrated to estimate log 2 fold-changes, p-values, and adjusted p-values. All data were normalized by equalizing median intensities, the summary method was Tukey's median polish, and the maximum quantile for deciding censored missing values was 0.999. Significantly dysregulated proteins were defined as those which had a fold change value >2 or <−2, with a p-value<0.05.

### Viral DNA Extraction and RT-PCR

DNA extraction was performed using the DNeasy Blood and Tissue Kit (Qiagen, Germantown, MD #69504). 1 μg of protein from VLV or KSHV preparations was mixed with 10 μg salmon sperm DNA (Thermo Fisher Scientific #15632011) as a carrier. Eluted DNA was adjusted to 100 ng/L and 1 μL was used for RT-PCR. RT-PCR was performed using a 10 μL reaction of the iTaq Universal SYBR Green Supermix (BioRad, Hercules, CA #1725120) with primers specific for the ORF50 gene (F: 5′-CGCAATGCGTTACGTTGTTG-3′ (SEQ ID NO: 4), R: 5′-GCCCGGACTGTTGAATCG-3′ (SEQ ID NO: 5)) on a 96-well CFX Connect Real-Time PCR system (BioRad). Cycle threshold (Ct) was compared to the threshold obtained using primers for the actin gene (F: 5′-CATGTACGTTGCTATCCAGGC-3′ (SEQ ID NO: 6), R: 5′-CTCCTTAATGTCACGCACGAT-3′ (SEQ ID NO: 7)) to normalize to the amount of carrier DNA isolated. ORF50/Actin ratio was calculated using 2\hat{ }ΔCt.

### KSHV Titer and Neutralization Assay

KSHV viral titer was obtained by infecting 10,000 HEK293 cells seeded overnight in a 96-well plate. 50 μL of virus diluted in serum-free DMEM was added to each well and incubated at 37° C. for 2 hours. For spin infection, an additional 50 μL of serum-free DMEM was added to each well and the plate was spun at 400 g for 20 minutes before the 2-hour incubation. The inoculum was removed, replaced with growth medium, and incubated for an additional 2 days. Infection was measured by flow cytometry for GFP+ cells on an Attune NxT with a plate autosampler (Thermo Fisher Scientific). Flow cytometry data was analyzed with FlowJo (BD, Ashland, OR).

KSHV neutralization was performed by infection of 10,000 HEK293 cells seeded overnight in a 96-well plate. The amount of virus used was calculated to give about 5% GFP+ cells for samples without neutralization. This amount differed between free virus and spin infection, as spin infected resulted in about 3× more infected cells from the same volume of virus. All assays comparing complement-enhanced neutralization were performed using spin infection. Immune serum was diluted in DMEM containing 2% normal mouse serum (Abcam, Cambridge, UK #ab7486) and KSHV was diluted in serum-free DMEM. For complement-dependent neutralization, KSHV was diluted with DMEM containing 2% guinea pig serum (Complement Technology Inc., Tyler, TX #GPS). Diluted serum was mixed with diluted virus at a 1:1 ratio and incubated at 37° C. for 1 hour. 50 μL of the serum/virus mixture was used to infect cells as described for the viral titer above. Neutralization was calculated as (% GFP_{PBS}−% GFPᵢₘₘᵤₙₑ)/% GFP_{PBS}*100, where % GFP_{PBS} is the percentage of cells expressing GFP from wells infected with virus mixed with control serum from mock-immunized mice and % GFPᵢₘₘᵤₙₑ is from experimental wells infected with virus mixed with serum from immunized mice. If % GFPᵢₘₘᵤₙₑ was greater than % GFP_{PBS}, neutralization was defined to be zero. NT50 was defined as the last serum dilution before neutralization drops below 50%.

### Mice and Immunizations

All animal experiments were conducted with the approval of the UCLA Institutional Animal Care and Use Committee and the Chancellor's Animal Research Committee. 6-10 week old female C57Bl/6J mice (The Jackson Laboratory, Bar Harbor, ME #000664) were immunized with PBS, VLV, or KSHV virions in a 50 μL volume intramuscularly or a 200 μL volume intraperitoneally using insulin syringes (Becton, Dickinson, and Company (BD), Franklin Lakes, NJ #329461) at the described time points. Immunogens were premixed with adjuvants and injected in the same volume described above. ODN2395 CpG adjuvant was purchased from Invivogen (#vac-2395-1). 21-mer polyU with phosphothioate linkages (polyUs) was custom synthesized by IDT. Lipid nanoparticles were prepared using a self-assembly process using methods in the art; the ionizable cationic lipid and LNP composition are described WO 2017/004143, which is herein incorporated by reference in its entirety. At the experimental endpoint, mice were euthanized and blood was collected by cardiac puncture with tuberculin syringes (BD #309623). Serum was collected by centrifugation in serum gel tubes (Sarstedt, Numbrecht, Germany #41.1378.005) and heat inactivated at 56° C. for 30 minutes before storage at −80° C. Splenocytes were harvested in RPMI (Corning #10040CV) containing 10% FBS and 1× PenStrep. Single cell suspensions were prepared by pushing spleens through 70 μm cell strainers (Fisher Scientific #22-363-548). Red blood cells were removed with ACK lysing buffer (Thermo Fisher Scientific #A1049201). Splenocytes were resuspended in complete RPMI and stored at 4° C. for no longer than overnight until stimulation.

### Bead-Based Multiplexed KSHV Antibody Assay

The assay was adapted to murine antibodies using methods in the art. Briefly, 68 recombinant KSHV antigens were each covalently attached to Bio-Plex Pro Magnetic COOH beads (Bio-Rad #MC10026 to #MC10065) via a sulfo-N-hydroxysulfosuccinimide mediated ester according to the manufacturer's protocol. enAntigens were split into two sets, with K8.1, ORF38, and ORF73 present in both sets to ensure consistency. 2500 beads in 50 μL assay buffer were added to each well and 50 μL of serum diluted 1:200 was added. Beads were incubated with serum for 1 hour at room temperature and washed with assay buffer. Beads were then incubated with an R-PE-labeled goat anti-mouse IgG secondary antibody (Jackson ImmunoResearch Laboratories, Inc., West Grove, PA #115-116-146) for 30 minutes. Samples were washed, resuspended in 100 μL assay buffer and analyzed on the Bio-Plex 200 system (Bio-Rad #171000201). The median fluorescence intensity (MFI) across all counted beads was computed for each sample and recorded after subtracting the background fluorescence.

### Enzyme-Linked Immunospot (ELISpot) Assay

ELISpot using VLV and KSHV+BPL stimulation was performed using a murine TNFα/IFNγ ELISpot kit (Cellular Technology Limited (CTL), Shaker Heights, OH #mIFNgTNFα-1M/10) following the manufacturer's instructions. Negative control wells contained unstimulated splenocytes and positive control wells were stimulated with a cocktail of phorbol 12-myristate 13-acetate (PMA) and ionomycin (Thermo Fisher Scientific #00-4970-03). Experimental wells were stimulated with 1 μg/mL VLV, VLV+BPL, or KSHV+BPL as indicated. 500,000 cells were used per well and plates were incubated for 20-22 hours at 37° C. before development. Plates were scanned and spot counts were analyzed by CTL. The number of spots in negative control wells was subtracted from experimental wells to determine the number of antigen-specific spot-forming cells.

### Activation Induced Marker Assay

250,000 splenocytes were plated in 100 μL complete RPMI in a 96-well U-bottom plate (Fisher Scientific #FB012932) and stimulated with the same conditions as for ELISpot. After 18-20 hours, cells were transferred to a 96-well V-bottom plate (Corning #3897) and washed with FACS buffer (DPBS containing 2% FBS and 0.05% sodium azide). All wash steps were followed by centrifugation at 500 g for 5 minutes to pellet cells. Cells were resuspended in 50 μL DPBS containing a 1:25 dilution of Fc block (anti-CD16/CD32 clone 93, Thermo Fisher Scientific #14-0161-86) and incubated at 4° C. for 10 minutes. 50 μL of cell surface antibodies were added to a final dilution of 1:200 and cells were further incubated for 30-60 minutes at 4° C. Cells were stained with the following antibody cocktail: anti-TCR beta-PerCP-Cy5.5 clone H57-597, CD4-PE-Cy7 clone RM4-5, CD8a-PE clone 53-6.7, CD69-FITC clone H1.2F3, OX40-Super Bright 780 clone OX-86, and 4-1BB-APC clone 17B5 (Thermo Fisher Scientific #50-158-64, 50-154-37, 50-112-9416, 50-965-3, 78-134-182, and 50-112-9043, respectively). After staining, cells were washed twice with FACS buffer, then fixed in 1% paraformaldehyde with incubation at 4° C. for 20 minutes. After fixation, cells were washed twice in FACS buffer then resuspended in FACS buffer. Cells were analyzed on an Attune NxT with a plate autosampler and data was analyzed using FlowJo. AIM+ cells were defined as OX40+4-1BB+ for CD4 T cells and CD69+4-1BB+ for CD8 T cells. Antigen-specific AIM+ cells were calculated by subtracting AIM+ cells in unstimulated samples from stimulated samples.

### KSHV ORFeome ELISpot

The ORFeome assay was adapted to murine using methods in the art. KSHV 15-mer peptides overlapping by 10 amino acids spanning the entire KSHV genome and totaling to over 7500 were synthesized using methods in the art (Mimotopes, Victoria, Australia). Peptide sequences were based on the sequence of BC-1 cell line derived virus. Eighty-five peptide pools were prepared, each corresponding to a single KSHV ORF except ORF64 which was represented by 3 pools. A total of 83 ORFs were represented. The individual lyophilized peptides were reconstituted using 50% acetonitrile. Peptide pools representing each ORF were prepared by combining the reconstituted peptides corresponding to that ORF. The pools were frozen and re-lyophilized. The lyophilized pools were reconstituted in DMSO (<20%) and PBS. A concentration of 5 μg/ml/peptide was used in the assay.

A single 96 well precoated mouse IFN-7 ELISpot plate (Mabtech, Cincinnati, OH #3321-4AST-2) was used per mouse to map responses to the 83 KSHV ORFs. Two positive controls, concanavalin A and cell stimulation Cocktail (Thermo Fisher Scientific) and one negative control, SIV Gag CM9 peptide (New England Peptide, Gardner, MA) were used. VLV was used as a positive control for VLV and KSHV vaccinated mice and a negative control for mock vaccinated mice. A medium only control was used to monitor background activity. All controls excluding the medium only well were plated in duplicate, the KSHV ORFs were plated in single wells and medium only controls were plated in triplicates. Each well of the ELISpot plate was seeded with 160,000-500,000 freshly processed splenocytes and incubated for 18 hours at 37° C., 5% CO₂. Two step detection with R4-6Δ2-biotin and Streptavidin-ALP, developed with BCIP/NBT-plus substrate was used and plates were read using the CTL ImmunoSpot Analyzer (CTL).

### K8.1 and ORF4 ELISA

ELISA plates were prepared by coating with recombinant K8.1 or ORF4 protein using methods in the art. Coated 96-well plates were blocked with assay buffer consisting of DPBS with 2.5% BSA (% w/v), 2.5% normal goat serum (% v/v) (Equitech-Bio, Kerrville, TX #SG-0500), 0.005% Tween-20 (% v/v) (Fisher Scientific #BP337-500), and 0.005% Triton X-100 (% v/v) (Fisher Scientific #BP151-500) and stored at −80° C. until use. All serum samples and secondary antibodies were diluted in assay buffer. Coated plates were thawed at room temperature on an orbital shaker and washed with DPBS containing 0.1% Tween-20 (% v/v) (PBS-T) twice for 3 minutes. Plates were then washed twice quickly with PBS-T before the addition of 50 μL serially diluted immune serum. 6-8 wells per plate were incubated with assay buffer containing no primary antibody as a background control. Plates were incubated for 1-2 hours at room temperature on an orbital shaker. Plates were washed with PBS-T twice for 3 minutes, then twice quickly before the addition of 50 μL 1:4000 goat anti-mouse HRP secondary antibody (Thermo Fisher Scientific #62-6520) or 1:5000 KPL peroxidase-labeled goat anti-human IgG (gamma) (LGC Clinical Diagnostics #474-1002). Secondary antibody was incubated for 1 hour at room temperature with shaking. Plates were then washed once with PBS-T for 3 minutes, then four times quickly. After one final wash with PBS (no Tween-20), 100 μL 1-Step Ultra TMB ELISA Substrate (Thermo Fisher Scientific #34028) was added to each well. Plates were covered to protect them from light and incubated at room temperature for 30 minutes with shaking. Signal development was stopped by the addition of 100 μL 1 M sulfuric acid (Sigma-Aldrich #1603131000) and the optical density at 450 nm (OD450) was measured with a ClarioStar plate reader (BMG Labtech, Cary, NC). Endpoint ELISA titer was defined as the first dilution before the OD450 dropped below the average signal from PBS-immunized serum at a 1:1000 dilution.

### Immunofluorescence Assay (IFA)

iSLK cells were analyzed 2 days post reactivation with 1-5 μg/mL doxycycline and 1 mM sodium butyrate and 293T cells were analyzed 1-2 days post transfection with plasmid of interest using BioT transfection reagent (Bioland Scientific LLC, Paramount, CA #B01). Cells were fixed with 4% paraformaldehyde (Electron Microscopy Sciences #15710) in DPBS for 15 minutes at room temperature without shaking. Cells were washed 3 times with PBS for 5 minutes and blocked with IFA buffer consisting of DPBS with 10% heat inactivated FBS (% v/v) and 3% BSA (% w/v) for 1 hour at room temperature with orbital shaking. For intracellular IFA, IFA buffer contained 0.3% Triton X-100 (% v/v). Cells were probed with primary antibody diluted in IFA buffer overnight at 4° C. on a rocker. After primary incubation, cells were washed 3 times with PBS for 5 minutes before the addition of 1:2000 goat anti-mouse IgG(H+L)-Alexa Fluor 594 secondary antibody (Thermo Fisher Scientific #A11032) diluted in IFA buffer. Cells were covered to protect them from light and incubated with secondary antibody for 2 hours at room temperature on an orbital shaker. Secondary antibody was removed, and nuclei were stained with 1:10000 aqueous Hoescht 33342 solution (Thermo Fisher Scientific #H3570) diluted in IFA buffer for 10 minutes at room temperature protected from light with orbital shaking. Cells were washed three times for 5 minutes with PBS before being visualized by fluorescence microscopy.

### Serum Antibody Removal by Cell Adsorption

293T cells were seeded in 10 cm tissue culture dishes (VWR #10062-880) at a density of 4×10⁶ cells. One day after seeding, cells were transfected using BioT with 10 g of plasmid for the glycoprotein of interest per dish. One day after transfection, cells were washed gently with DPBS, pushed into suspension with DPBS, and split into 3 tubes. Cells were pelleted and kept at 4° C. for no longer than 1-2 days before use. Serum of interest was diluted 1:2 with DPBS and 50 μL of the diluted serum was used to resuspend the cell pellet. The mixture was inverted at room temperature for 4 hours before the cells were removed by centrifugation at 500 g for 5 minutes. This was repeated twice for a total of 3 adsorptions per serum sample per cell line. The removal of glycoprotein-specific antibodies was verified by immunofluorescence.

### Statistical Analysis

Statistical analysis was performed using the tests described in the figure legends using Prism (Graphpad Software, San Diego, CA).

### Additional Embodiments

Embodiment 1: A composition for inducing and/or enhancing complement-mediated neutralization of a herpesvirus which comprises, consists essentially of, or consists of
(1)(a) a mutant of the herpesvirus, which is a capsid deficient mutant that is deficient in the major capsid protein or is a gB mutant that has a mutation in its glycoprotein B (gB), (b) virus-like vesicles made from a capsid deficient mutant of the herpesvirus, (c) a protein that comprises, consists essentially of, or consists of amino acid residues 24-150 of an ORF4 protein of the herpesvirus or a nucleic acid molecule that encodes the protein, and/or (d) an anti-ORF4 antibody raised against (a), (b), or (c) above or a synthetic antibody derived from the anti-ORF4 antibody; and
(2) an adjuvant.

Embodiment 2: The composition according to Embodiment 1, wherein the adjuvant is a CpG oligonucleotide (CpG ODN) that is an agonist of toll like receptor 9 (TLR9), an empty lipid nanoparticle (LNP), or a polyU oligonucleotide encapsulated in a lipid nanoparticle (polyUs LNP).

Embodiment 3: The composition according to Embodiment 2, wherein the polyU oligonucleotide
(a) is 10-25 bases long; and/or
(b) has phosphorothioate linkages.

Embodiment 4: The composition according to any one of Embodiments 1-3, wherein the adjuvant is a 21-mer phosphorothioate-linked polyU oligonucleotide.

Embodiment 5: The composition according to any one of Embodiments 1-4, wherein the herpesvirus is a Kaposi's Sarcoma Associated Herpesvirus (KSHV).

Embodiment 6: The composition according to Embodiment 5, wherein the capsid deficient mutant lacks an ORF25 gene or its ORF25 gene encodes an ORF25 protein that lacks at least 25 amino acid residues at the N-terminus as compared to the ORF25 wildtype sequence.

Embodiment 7: The composition according to Embodiment 5 or Embodiment 6, wherein the ORF25 protein has a mutation selected from ORF25Δ30, ORF25Δ31, ORF25Δ32, ORF25Δ33, ORF25Δ34, ORF25Δ35, ORF25Δ36, ORF25Δ37, ORF25Δ38, ORF25Δ39, ORF25Δ40, ORF25Δ41, ORF25Δ42, ORF25Δ43, ORF25Δ44, ORF25Δ45, ORF25Δ46, ORF25Δ47, ORF25Δ48, ORF25Δ49, ORF25Δ50, ORF25Δ51, ORF25Δ52, ORF25Δ53, ORF25Δ54, ORF25Δ55, ORF25Δ56, ORF25Δ57, ORF25Δ58, ORF25Δ59, ORF25Δ60, ORF25Δ61, ORF25Δ62, ORF25Δ63, ORF25Δ64, and ORF25Δ65 as compared to the ORF25 wildtype sequence.

Embodiment 8: The composition according to any one of Embodiments 1-7, wherein the protein comprises amino acid residues 24 314 of the ORF4 of the herpesvirus.

Embodiment 9: The composition according to any one of Embodiments 1-8, wherein the ORF4 of the herpesvirus comprises at least 95%, at least 96%, at least 97%, at least 98%, at least 99%, or 100% sequence identity to the ORF4 wildtype sequence of KSHV.

Embodiment 10: A method of immunizing a subject against infection by a herpesvirus, which comprises administering to the subject a composition according to any one of Embodiments 1-9.

Embodiment 11: A method of inducing or enhancing an immune response against a herpesvirus in a subject, which comprises administering to the subject a composition according to any one of Embodiments 1-9.

Embodiment 12: The method according to Embodiment 11, wherein the immune response is complement-mediated neutralization of the herpesvirus.

Embodiment 13: A method of inhibiting or treating a herpesvirus infection and/or a disease caused by the herpesvirus infection in a subject, which comprises administering to the subject a composition according to any one of Embodiments 1-9.

### REFERENCES

The following references are herein incorporated by reference in their entirety with the exception that, should the scope and meaning of a term conflict with a definition explicitly set forth herein, the definition explicitly set forth herein controls:
Agrawal et al. (2017) Complement Evasion Strategies of Viruses: An Overview. Front Microbiol. 8:1117.
Alameh et al. (2021) Lipid nanoparticles enhance the efficacy of mRNA and protein subunit vaccines by inducing robust T follicular helper cell and humoral responses. Immunity. 54(12):2877-2892.e7.
Awasthi et al. (2011) Immunization with a vaccine combining herpes simplex virus 2 (HSV-2) glycoprotein C (gC) and gD subunits improves the protection of dorsal root ganglia in mice and reduces the frequency of recurrent vaginal shedding of HSV-2 DNA in guinea pigs compared to immunization with gD alone. J Virol. 85(20):10472-86.
Awasthi et al. (2019) Nucleoside-modified mRNA encoding HSV-2 glycoproteins C, D, and E prevents clinical and subclinical genital herpes. Sci Immunol. 4(39):eaaw7083.
Awasthi et al. (2009) Immunization with HSV-1 glycoprotein C prevents immune evasion from complement and enhances the efficacy of an HSV-1 glycoprotein D subunit vaccine. Vaccine. 27(49):6845-53.
Barasa et al. (2017) BALB/c mice immunized with a combination of virus-like particles incorporating Kaposi sarcoma-associated herpesvirus (KSHV) envelope glycoproteins gpK81, gB, and gH/gL induced comparable serum neutralizing antibody activity to UV-inactivated KSHV. Oncotarget. 8(21):34481-34497.
Bauer et al. (2001) Human TLR9 confers responsiveness to bacterial DNA via species-specific CpG motif recognition. PNAS USA. 98(16):9237-42.
Bernstein et al. (2016) Safety and efficacy of a cytomegalovirus glycoprotein B (gB) vaccine in adolescent girls: A randomized clinical trial. Vaccine. 34(3):313-9.
Birkmann et al. (2001) Cell surface heparan sulfate is a receptor for human herpesvirus 8 and interacts with envelope glycoprotein K81. J Virol. 75(23):11583-93.
Boldogh et al. (1996) Kaposi's sarcoma herpesvirus-like DNA sequences in the saliva of individuals infected with human immunodeficiency virus. Clin Infect Dis. 23(2):406-7.
Bowyer et al. (2018) Activation-induced Markers Detect Vaccine-Specific CD4⁺ T Cell Responses Not Measured by Assays Conventionally Used in Clinical Trials. Vaccines (Basel). 6(3):50.
Brayfield et al. (2004) Distribution of Kaposi sarcoma-associated herpesvirus/human herpesvirus 8 in maternal saliva and breast milk in Zambia: implications for transmission. J Infect Dis. 189(12):2260-70.
Bauer et al. (2001) Human TLR9 confers responsiveness to bacterial DNA via species-specific CpG motif recognition. PNAS USA. 98(16):9237-42.
Britt et al. (1990) Cell surface expression of human cytomegalovirus (HCMV) gp55-116 (gB): use of HCMV-recombinant vaccinia virus-infected cells in analysis of the human neutralizing antibody response. J Virol. 64(3):1079-85.
Bruderer et al. (2017) Optimization of Experimental Parameters in Data-Independent Mass Spectrometry Significantly Increases Depth and Reproducibility of Results. Mol Cell Proteomics. 16(12):2296-2309.
Brulois et al. (2012) Construction and manipulation of a new Kaposi's sarcoma-associated herpesvirus bacterial artificial chromosome clone. J Virol.
86(18):9708-20.
Brulois et al. (2012) Construction and manipulation of a new Kaposi's sarcoma-associated herpesvirus bacterial artificial chromosome clone. J Virol. 86(18):9708-20.
Bu et al. (2019) Immunization with Components of the Viral Fusion Apparatus Elicits Antibodies That Neutralize Epstein-Barr Virus in B Cells and Epithelial Cells. Immunity. 50(5):1305-1316.e6.
Butler et al. (2009) Kaposi sarcoma-associated herpesvirus (KSHV) seroprevalence in population-based samples of African children: evidence for at least 2 patterns of KSHV transmission. J Infect Dis. 200(3):430-8.
Cesarman et al. (2019) Kaposi sarcoma. Nat Rev Dis Primers. 5(1):9.
Chang et al. (2009) Non-human primate model of Kaposi's sarcoma-associated herpesvirus infection. PLoS Pathog. 5(10):e1000606.
Corbett et al. (2020) SARS-CoV-2 mRNA vaccine design enabled by prototype pathogen preparedness. Nature. 586(7830):567-571.
Crabtree et al. (2017) Association of Household Food- and Drink-Sharing Practices With Human Herpesvirus 8 Seroconversion in a Cohort of Zambian Children. J Infect Dis. 216(7):842-849.
Dai et al. (2018) Structure and mutagenesis reveal essential capsid protein interactions for KSHV replication. Nature. 553(7689):521-525.
Dai et al. (2018) Structure and mutagenesis reveal essential capsid protein interactions for KSHV replication. Nature. 553(7689):521-525.
Dedicoat et al. (2004) Mother-to-child transmission of human herpesvirus-8 in South Africa. J Infect Dis. 190(6):1068-75.
Diebold et al. (2006) Nucleic acid agonists for Toll-like receptor 7 are defined by the presence of uridine ribonucleotides. Eur J Immunol. 36(12):3256-67.
Dittmer et al. (2016) Kaposi sarcoma-associated herpesvirus: immunobiology, oncogenesis, and therapy. J Clin Invest. 126(9):3165-75.
Dollery S J (2019) Towards Understanding KSHV Fusion and Entry. Viruses. 11(11):1073.
Dunkelberger & Song (2010) Complement and its role in innate and adaptive immune responses. Cell Res. 20(1):34-50.
Egan et al. (2020) An HSV-2 nucleoside-modified mRNA genital herpes vaccine containing glycoproteins gC, gD, and gE protects mice against HSV-1 genital lesions and latent infection. PLoS Pathog. 16(7):e1008795.
Elias & Gygi (2007) Target-decoy search strategy for increased confidence in large-scale protein identifications by mass spectrometry. Nat Methods. 4(3):207-14.
Ferlay et al. (2010) Estimates of worldwide burden of cancer in 2008: GLOBOCAN 2008. Int J Cancer. 127(12):2893-917.
Friedman et al. (1996) Immune evasion properties of herpes simplex virus type 1 glycoprotein gC. J Virol. 70(7):4253-60.
Ganem D (2010) KSHV and the pathogenesis of Kaposi sarcoma: listening to human biology and medicine. J Clin Invest. 120(4):939-49.
Gangappa et al. (2002) Antibody to a lytic cycle viral protein decreases gammaherpesvirus latency in B-cell-deficient mice. J Virol. 76(22):11460-8.
Geeraedts et al. (2008) Superior immunogenicity of inactivated whole virus H5N1 influenza vaccine is primarily controlled by Toll-like receptor signalling. PLoS Pathog. 4(8):e1000138.
Gong et al. (2017) Virus-Like Vesicles of Kaposi's Sarcoma-Associated Herpesvirus Activate Lytic Replication by Triggering Differentiation Signaling. J Virol. 91(15):e00362-17.
Grifoni et al. (2020) Targets of T Cell Responses to SARS-CoV-2 Coronavirus in Humans with COVID-19 Disease and Unexposed Individuals. Cell. 181(7):1489-1501.e15.
Hahn et al. (2009) Kaposi's sarcoma-associated herpesvirus gH/gL: glycoprotein export and interaction with cellular receptors. J Virol. 83(1):396-407.
Higgins et al. (2021) SARS-CoV-2 hijacks p38B/MAPK11 to promote viral protein translation. bioRxiv 457146.
Hjalgrim et al. The epidemiology of EBV and its association with malignant disease. In: Arvin A, Campadelli-Fiume G, Mocarski E, Moore P S, Roizman B, Whitley R,
Yamanishi K, editors. Human Herpesviruses: Biology, Therapy, and Immunoprophylaxis. Cambridge: Cambridge University Press; 2007. Chapter 53.
Jing et al. (2012) Cross-presentation and genome-wide screening reveal candidate T cells antigens for a herpes simplex virus type 1 vaccine. J Clin Invest. 122(2):654-73.
Johnson & Huber (2002) Directed egress of animal viruses promotes cell-to-cell spread. J Virol. 76(1):1-8.
Kalantari-Dehaghi et al. (2012) Discovery of potential diagnostic and vaccine antigens in herpes simplex virus 1 and 2 by proteome-wide antibody profiling. J Virol. 86(8):4328-39.
Kapadia et al. (2002) Critical role of complement and viral evasion of complement in acute, persistent, and latent gamma-herpesvirus infection. Immunity. 17(2):143-55.
Kedes et al. (1996) The seroepidemiology of human herpesvirus 8 (Kaposi's sarcoma-associated herpesvirus): distribution of infection in KS risk groups and evidence for sexual transmission. Nat Med. 2(8):918-24.
Koelle et al. (1997) Frequent detection of Kaposi's sarcoma-associated herpesvirus (human herpesvirus 8) DNA in saliva of human immunodeficiency virus-infected men: clinical and immunologic correlates. J Infect Dis. 176(1):94-102.
Kotturi et al. (2009) Of mice and humans: how good are HLA transgenic mice as a model of human immune responses? Immunome Res. 5:3.
Krishnan et al. (2004) Concurrent expression of latent and a limited number of lytic genes with immune modulation and antiapoptotic function by Kaposi's sarcoma-associated herpesvirus early during infection of primary endothelial and fibroblast cells and subsequent decline of lytic gene expression. J Virol. 78(7):3601-20.
Krogh et al. (2001) Predicting transmembrane protein topology with a hidden Markov model: application to complete genomes. J Mol Biol. 305(3):567-80.
Labo et al. (2014) Heterogeneity and breadth of host antibody response to KSHV infection demonstrated by systematic analysis of the KSHV proteome. PLoS Pathog. 10(3):e1004046.
Labo et al. (2019) Mutual detection of Kaposi's sarcoma-associated herpesvirus and Epstein-Barr virus in blood and saliva of Cameroonians with and without Kaposi's sarcoma. Int J Cancer. 145(9):2468-2477.
Lambert et al. (2006) Differences in the frequency and function of HHV8-specific CD8 T cells between asymptomatic HHV8 infection and Kaposi sarcoma. Blood. 108(12):3871-80.
Lawler & Stevenson (2020) A CD4+ T Cell-NK Cell Axis of Gammaherpesvirus Control. J Virol. 94(3):e01545-19.
Li et al. (2017) Complement enhances in vitro neutralizing potency of antibodies to human cytomegalovirus glycoprotein B (gB) and immune sera induced by gB/MF59 vaccination. NPJ Vaccines. 2:36.
Li et al. (2021) A conditionally replication-defective cytomegalovirus vaccine elicits potent and diverse functional monoclonal antibodies in a phase I clinical trial. NPJ Vaccines. 6(1):79.
Liu et al. (2021) Prefusion structure of human cytomegalovirus glycoprotein B and structural basis for membrane fusion. Sci Adv. 7(10):eabf3178.
Liu et al. (2018) Global epidemiology of human herpesvirus 8 in men who have sex with men: A systematic review and meta-analysis. J Med Virol. 90(3):582-591.
Maier et al. (2013) Biodegradable lipids enabling rapidly eliminated lipid nanoparticles for systemic delivery of RNAi therapeutics. Mol Ther. 21(8):1570-8.
Man et al. (1994) Diversity and dominance among TCR recognizing HLA-A21+ influenza matrix peptide in human MHC class I transgenic mice. J Immunol. 153(10):4458-67.
Mark et al. (2006) The Kaposi's sarcoma-associated herpesvirus complement control protein (KCP) binds to heparin and cell surfaces via positively charged amino acids in CCP1-2. Mol Immunol. 43(10):1665-75.
Mark et al. (2008) Separation of decay-accelerating and cofactor functional activities of Kaposi's sarcoma-associated herpesvirus complement control protein using monoclonal antibodies. Immunology. 123(2):228-38.
Martin et al. (1998) Sexual transmission and the natural history of human herpesvirus 8 infection. N Engl J Med. 338(14):948-54.
McHugh et al. (2017) Persistent KSHV Infection Increases EBV-Associated Tumor Formation In Vivo via Enhanced EBV Lytic Gene Expression. Cell Host Microbe. 22(1):61-73.e7.
Mellors et al. (2020) Viral Evasion of the Complement System and Its Importance for Vaccines and Therapeutics. Front Immunol. 11:1450.
Minab et al. (2021) Maternal Epstein-Barr Virus-Specific Antibodies and Risk of Infection in Ugandan Infants. J Infect Dis. 223(11):1897-1904.
Minhas & Wood (2014) Epidemiology and transmission of Kaposi's sarcoma-associated herpesvirus. Viruses. 6(11):4178-94.
Moradi et al. (2021) Hepatitis B core-based virus-like particles: A platform for vaccine development in plants. Biotechnol Rep (Amst). 29:e00605.
Mulama et al. (2019) A multivalent Kaposi sarcoma-associated herpesvirus-like particle vaccine capable of eliciting high titers of neutralizing antibodies in immunized rabbits. Vaccine. 37(30):4184-4194.
Nalwoga et al. (2021) Kaposi's sarcoma-associated herpesvirus T cell responses in HIV seronegative individuals from rural Uganda. Nat Commun. 12(1):7323.
Nalwoga et al. (2021) Kaposi's sarcoma-associated herpesvirus T cell responses in HIV seronegative individuals from rural Uganda. Nat Commun. 12(1):7323.
Nalwoga et al. (2020) Variation in KSHV prevalence between geographically proximate locations in Uganda. Infect Agent Cancer. 15:49.
Ndeupen et al. (2021) The mRNA-LNP platform's lipid nanoparticle component used in preclinical vaccine studies is highly inflammatory. iScience. 24(12):103479.
Nelson et al. (2018) HCMV glycoprotein B subunit vaccine efficacy mediated by nonneutralizing antibody effector functions. PNAS USA. 115(24):6267-6272.
Nielsen H (1905) Predicting Secretory Proteins with SignalP. Methods Mol Biol. 1611:59-73.
Okroj et al. (2007) Antibodies against Kaposi sarcoma-associated herpes virus (KSHV) complement control protein (KCP) in infected individuals. Vaccine. 25(48):8102-9.
Okroj et al. (2011) Prevalence of antibodies against Kaposi's sarcoma associated herpes virus (KSHV) complement inhibitory protein (KCP) in KSHV-related diseases and their correlation with clinical parameters. Vaccine. 29(6):1129-34.
Olp et al. (2013) Early childhood infection of Kaposi's sarcoma-associated herpesvirus in Zambian households: a molecular analysis. Int J Cancer. 132(5):1182-90.
Pallesen et al. (2017) Immunogenicity and structures of a rationally designed prefusion MERS-CoV spike antigen. PNAS USA. 114(35):E7348-E7357.
Pavlova et al. (2013) An Epstein-Barr virus mutant produces immunogenic defective particles devoid of viral DNA. J Virol. 87(4):2011-22.
Perez-Riverol et al. (2022) The PRIDE database resources in 2022: a hub for mass spectrometry-based proteomics evidences. Nucleic Acids Res. 50(D1):D543-D552.
Pertel P E (2002) Human herpesvirus 8 glycoprotein B (gB), gH, and gL can mediate cell fusion. J Virol. 76(9):4390-400.
Petersen et al. (2011) SignalP 40: discriminating signal peptides from transmembrane regions. Nat Methods. 8(10):785-6.
Plotkin S A (2010) Correlates of protection induced by vaccination. Clin Vaccine Immunol. 17(7):1055-65.
Poppe et al. (2020) Relationships Between Maternal Antibody Responses and Early Childhood Infection With Kaposi Sarcoma-Associated Herpesvirus. J Infect Dis. 222(10):1723-1730.
Pulendran et al. (2021) Emerging concepts in the science of vaccine adjuvants. Nat Rev Drug Discov. 20(6):454-475.
Reed et al. (2013) Key roles of adjuvants in modern vaccines. Nat Med. 19(12):1597-608.
Reiss et al. (2017) Comparative analysis of activation induced marker (AIM) assays for sensitive identification of antigen-specific CD4 T cells. PLoS One. 12(10):e0186998.
Robey et al. (2009) The CD8 and CD4 T-cell response against Kaposi's sarcoma-associated herpesvirus is skewed towards early and late lytic antigens. PLoS One. 4(6):e5890.
Robey et al. (1905) The T-Cell Immune Response against Kaposi's Sarcoma-Associated Herpesvirus. Adv Virol. 2010:340356.
Roshan et al. (2017) T-cell responses to KSHV infection: a systematic approach. Oncotarget. 8(65):109402-109416.
Ruiss et al. (2011) A virus-like particle-based Epstein-Barr virus vaccine. J Virol. 85(24):13105-13.
Sabourin et al. (2021) Malaria Is Associated With Kaposi Sarcoma-Associated Herpesvirus Seroconversion in a Cohort of Western Kenyan Children. J Infect Dis. 224(2):303-311.
Shirai et al. (2020) Lipid Nanoparticle Acts as a Potential Adjuvant for Influenza Split Vaccine without Inducing Inflammatory Responses. Vaccines (Basel). 8(3):433.
Spiller et al. (2003) Complement regulation by Kaposi's sarcoma-associated herpesvirus ORF4 protein. J Virol. 77(1):592-9.
Spiller et al. (2003) Functional activity of the complement regulator encoded by Kaposi's sarcoma-associated herpesvirus. J Biol Chem. 278(11):9283-9.
Swain et al. (2012) Expanding roles for CD4⁺ T cells in immunity to viruses. Nat Rev Immunol. 12(2):136-48.
Swaminathan et al. (2016) A novel lipid nanoparticle adjuvant significantly enhances B cell and T cell responses to sub-unit vaccine antigens. Vaccine. 34(1):110-9.
Tahtinen et al. (2022) IL-1 and IL-1ra are key regulators of the inflammatory response to RNA vaccines. Nat Immunol. 23(4):532-542.
Tischer et al. (1905) En passant mutagenesis: a two step markerless red recombination system. Methods Mol Biol. 634:421-30.
Uppal et al. (2014) KSHV LANA—the master regulator of KSHV latency. Viruses. 6(12):4961-98.
Van et al. (2018) Immunogenic particles with a broad antigenic spectrum stimulate cytolytic T cells and offer increased protection against EBV infection ex vivo and in mice. PLoS Pathog. 14(12):e1007464.
Vollmer et al. (2020) The prefusion structure of herpes simplex virus glycoprotein B. Sci Adv. 6(39):eabc1726.
Wang et al. (2001) Primary human herpesvirus 8 infection generates a broadly specific CD8(+) T-cell response to viral lytic cycle proteins. Blood. 97(8):2366-73.
Wang et al. (2001) Human herpesvirus 8 envelope glycoprotein K81A interaction with the target cells involves heparan sulfate. J Virol. 75(16):7517-27.
Wang & Roden (2013) Virus-like particles for the prevention of human papillomavirus-associated malignancies. Expert Rev Vaccines. 12(2):129-41.
Wang et al. (2014) Humanized-BLT mouse model of Kaposi's sarcoma-associated herpesvirus infection. PNAS USA. 111(8):3146-51.
Webster-Cyriaque et al. (1997) Epstein-Barr virus and human herpesvirus 8 prevalence in human immunodeficiency virus-associated oral mucosal lesions. J Infect Dis. 175(6):1324-32.
Woodle et al. (2001) Kaposi sarcoma: an analysis of the US and international experiences from the Israel Penn International Transplant Tumor Registry. Transplant Proc. 33(7-8):3660-1.
WO2017004143

All scientific and technical terms used in this application have meanings commonly used in the art unless otherwise specified.

As used herein, the terms “subject”, “patient”, and “individual” are used interchangeably to refer to humans and non-human animals. The terms “non-human animal” and “animal” refer to all non-human vertebrates, e.g., non-human mammals and non-mammals, such as non-human primates, horses, sheep, dogs, cows, pigs, chickens, and other veterinary subjects and test animals. In some embodiments, the subject is a mammal. In some embodiments, the subject is a human.

The use of the singular can include the plural unless specifically stated otherwise. As used in the specification and the appended claims, the singular forms “a”, “an”, and “the” can include plural referents unless the context clearly dictates otherwise.

As used herein, “and/or” means “and” or “or”. For example, “A and/or B” means “A, B, or both A and B” and “A, B, C, and/or D” means “A, B, C, D, or a combination thereof” and said “A, B, C, D, or a combination thereof” means any subset of A, B, C, and D, for example, a single member subset (e.g., A or B or C or D), a two-member subset (e.g., A and B; A and C; etc.), or a three-member subset (e.g., A, B, and C; or A, B, and D; etc.), or all four members (e.g., A, B, C, and D).

As used herein, the phrase “one or more of”, e.g., “one or more of A, B, and/or C” means “one or more of A”, “one or more of B”, “one or more of C”, “one or more of A and one or more of B”, “one or more of B and one or more of C”, “one or more of A and one or more of C” and “one or more of A, one or more of B, and one or more of C”.

As used herein, the phrase “consists essentially of” in the context of the recited ingredient(s) in a composition, means that the composition may include additional ingredients so long as the additional ingredients do not adversely impact the activity, e.g., biological or pharmaceutical function, of the recited ingredient(s).

The phrase “comprises, consists essentially of, or consists of A” is used as a tool to avoid excess page and translation fees and means that in some embodiments the given thing at issue: comprises A, consists essentially of A, or consists of A. For example, the sentence “In some embodiments, the composition comprises, consists essentially of, or consists of A” is to be interpreted as if written as the following three separate sentences: “In some embodiments, the composition comprises A. In some embodiments, the composition consists essentially of A. In some embodiments, the composition consists of A.”

Similarly, a sentence reciting a string of alternates is to be interpreted as if a string of sentences were provided such that each given alternate was provided in a sentence by itself. For example, the sentence “In some embodiments, the composition comprises A, B, or C” is to be interpreted as if written as the following three separate sentences: “In some embodiments, the composition comprises A. In some embodiments, the composition comprises B. In some embodiments, the composition comprises C.” As another example, the sentence “In some embodiments, the composition comprises at least A, B, or C” is to be interpreted as if written as the following three separate sentences: “In some embodiments, the composition comprises at least A. In some embodiments, the composition comprises at least B. In some embodiments, the composition comprises at least C.”

As used herein, the terms “protein”, “polypeptide” and “peptide” are used interchangeably to refer to two or more amino acids linked together. Groups or strings of amino acid abbreviations are used to represent peptides. Except when specifically indicated, peptides are indicated with the N-terminus on the left and the sequence is written from the N-terminus to the C-terminus. Except when specifically indicated, peptides are indicated with the N-terminus on the left and the sequences are written from the N-terminus to the C-terminus. Similarly, except when specifically indicated, nucleic acid sequences are indicated with the 5′ end on the left and the sequences are written from 5′ to 3′.

As used herein, a given percentage of “sequence identity” refers to the percentage of nucleotides or amino acid residues that are the same between sequences, when compared and optimally aligned for maximum correspondence over a given comparison window, as measured by visual inspection or by a sequence comparison algorithm in the art, such as the BLAST algorithm, which is described in Altschul et al., (1990) J Mol Biol 215:403-410. Software for performing BLAST (e.g., BLASTP and BLASTN) analyses is publicly available through the National Center for Biotechnology Information (ncbi.nlm.nih.gov). The comparison window can exist over a given portion, e.g., a functional domain, or an arbitrarily selection a given number of contiguous nucleotides or amino acid residues of one or both sequences. Alternatively, the comparison window can exist over the full length of the sequences being compared. For purposes herein, where a given comparison window (e.g., over 80% of the given sequence) is not provided, the recited sequence identity is over 100% of the given sequence. Additionally, for the percentages of sequence identity of the proteins provided herein, the percentages are determined using BLASTP 2.8.0+, scoring matrix BLOSUM62, and the default parameters available at blast.ncbi.nlm.nih.gov/Blast.cgi. See also Altschul, et al., (1997) Nucleic Acids Res 25:3389-3402; and Altschul, et al., (2005) FEBS J 272:5101-5109.

Optimal alignment of sequences for comparison can be conducted, e.g., by the local homology algorithm of Smith & Waterman, Adv Appl Math 2:482 (1981), by the homology alignment algorithm of Needleman & Wunsch, J Mol Biol 48:443 (1970), by the search for similarity method of Pearson & Lipman, PNAS USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by visual inspection.

To the extent necessary to understand or complete the disclosure of the present invention, all publications, patents, and patent applications mentioned herein are expressly incorporated by reference therein to the same extent as though each were individually so incorporated.

Having thus described exemplary embodiments of the present invention, it should be noted by those skilled in the art that the within disclosures are exemplary only and that various other alternatives, adaptations, and modifications may be made within the scope of the present invention. Accordingly, the present invention is not limited to the specific embodiments as illustrated herein, but is only limited by the following claims.

What is claimed is:

1. A composition for inducing and/or enhancing complement-mediated neutralization of a herpesvirus which comprises, consists essentially of, or consists of
(1)(a) a mutant herpesvirus is a capsid deficient mutant that has a deletion in its major capsid protein as compared to the wildtype major capsid protein and/or a gB mutant having a mutation in its glycoprotein B (gB); (b) virus-like vesicles made from the mutant herpesvirus which is capsid deficient; (c) a protein that comprises, consists essentially of, or consists of amino acid residues 24-150 of an ORF4 protein of the herpesvirus or a nucleic acid molecule that encodes the protein; and/or (d) an antibody raised against (a), (b), or (c), or a synthetic antibody derived from the antibody; and
(2) an adjuvant.

2. The composition according to claim 1, wherein the gB protein of the mutant herpesvirus has (1) a sequence that is at least 95%, at least 96%, at least 97%, at least 98%, or at least 99% sequence identity to SEQ ID NO: 1, and (2) an amino acid other than aspartic acid (D) at position 470 and/or an amino acid other than glycine (G) at position 471 when optimally aligned thereto.

3. The composition according to claim 1, wherein the composition comprising the virus-like vesicles is free of infectious virions.

4. The composition according to claim 1, wherein the adjuvant is a CpG oligonucleotide (CpG ODN) that is an agonist of toll-like receptor 9 (TLR9), an empty lipid nanoparticle (LNP), or a polyU oligonucleotide encapsulated in a lipid nanoparticle (polyUs-LNP).

5. The composition according to claim 4, wherein the polyU oligonucleotide
(a) is 10-25 bases long; and/or
(b) has phosphorothioate linkages.

6. The composition according to claim 1, wherein the adjuvant is a 21-mer phosphorothioate-linked polyU oligonucleotide.

7. The composition according to claim 1, wherein the herpesvirus is a Kaposi's Sarcoma Associated Herpesvirus (KSHV).

8. The composition according to claim 7, wherein the capsid deficient mutant lacks an ORF25 gene or its ORF25 gene encodes an ORF25 protein that lacks at least 25 amino acid residues at the N-terminus as compared to the ORF25 wildtype sequence.

9. The composition according to claim 7, wherein the ORF25 protein has a mutation selected from ORF25Δ30, ORF25Δ31, ORF25Δ32, ORF25Δ33, ORF25Δ34, ORF25Δ35, ORF25Δ36, ORF25Δ37, ORF25Δ38, ORF25Δ39, ORF25Δ40, ORF25Δ41, ORF25Δ42, ORF25Δ43, ORF25Δ44, ORF25Δ45, ORF25Δ46, ORF25Δ47, ORF25Δ48, ORF25Δ49, ORF25Δ50, ORF25Δ51, ORF25Δ52, ORF25Δ53, ORF25Δ54, ORF25Δ55, ORF25Δ56, ORF25Δ57, ORF25Δ58, ORF25Δ59, ORF25Δ60, ORF25Δ61, ORF25Δ62, ORF25Δ63, ORF25Δ64, and ORF25Δ65 as compared to the ORF25 wildtype sequence.

10. The composition according to claim 1, wherein the protein comprises or consists of amino acid residues 24-314 of the ORF4 of the herpesvirus.

11. The composition according to claim 10, wherein the ORF4 of the herpesvirus contains at least 95%, at least 96%, at least 97%, at least 98%, at least 99%, or 100% sequence identity to the ORF4 wildtype sequence of KSHV.

12. A method of making an antibody against a herpesvirus, which comprises immunizing a subject with the composition according to claim 1, wherein the composition comprises the mutant herpesvirus, the virus-like vesicles, and/or the protein and the adjuvant.

13. The method according to claim 12, wherein the antibody is a complement-mediated neutralizing antibody.

14. Sera comprising antibodies against a herpesvirus made by the method according to claim 12 and isolating the sera from the subject.

15. A method of immunizing a subject against infection by a herpesvirus, which comprises administering to the subject a composition according to claim 1.

16. A method of inducing or enhancing an immune response against a herpesvirus in a subject, which comprises administering to the subject a composition according to claim 1.

17. The method according to claim 16, wherein the immune response is complement-mediated neutralization of the herpesvirus.

18. A method of inhibiting or treating a herpesvirus infection and/or a disease caused by the herpesvirus infection in a subject, which comprises administering to the subject a composition according to claim 1.

19. The method according to claim 18, wherein the disease is a cancer.

20. The method according to claim 19, wherein the cancer is Kaposi's Sarcoma (KS) or primary effusion lymphoma.
